(19) United States
(12) Patent Application Publication
(10) Pub. No.: US 2014/0248291 A1
(43) Pub. Date: Sep. 4, 2014

(54) Modulation of Cellular Migration

(71) Applicant: THE UAB RESEARCH FOUNDATION, Birmingham, AL (US)

(72) Inventors: Harald W. Sontheimer, Birmingham, AL (US); Vedrana Montana Parpura, Birmingham, AL (US)

(73) Assignee: The UAB Research Foundation, Birmingham, AL (US)

(21) Appl. No.: 14/008,979

(22) PCT Filed: Mar. 30, 2012
(86) PCT No.: PCT/US12/31573

§ 371 Date: Apr. 8, 2014

Related U.S. Application Data
(60) Provisional application No. 61/469,649, filed on Mar. 30, 2011.

(51) Int. Cl. A61K 39/395; G01N 33/574; A61K 31/713

(52) U.S. Cl. CPC A61K 39/39558; A61K 31/713; G01N 33/574
USPC 424/174.1; 514/44 A; 435/375; 435/7.1

(57) ABSTRACT

Methods, kits, and compositions are provided for addressing cancer through the interaction of bradykinin (BK) and the bradykinin-2-receptor (B2R). This interaction controls cellular invasion, as has been unexpectedly observed in glioma cells, A composition is provided for the treatment of cancer by disrupting this interaction using an inhibitor or BK or B2R that can. be administered to the subject. Diagnostic processes are provided, involving measuring levels of BK or B2R to determine the potential for cancer (or to determine the invasive potential of a given cancer). Modulators of BK. and B2R may be used to modulate cellular migration, both in vivo and in vitro. Potential modulators of cellular migration can be screened by measuring the effect of the potential modulator on BK or B2R,

### STATEMENT OF GOVERNMENT FUNDING

The subject matter described herein was at least partially made with government support under grant numbers NS031234, NS036692, and NS052634 awarded by the U.S. Department of Health and Human Services, National Institutes of Health. The government of the United States has certain rights in the subject matter described herein.

### BACKGROUND

A. Field of the Disclosure

The present disclosure relates generally to the modulation of cell migration. Specific compounds, as well as methods, cells, and compositions for use therewith are provided.

B. Background

Gliomas derive from glial cells or their precursors and are the most common malignant primary brain tumors. Gliomas have an extraordinary ability to infiltrate the healthy brain (Laerum et al., 1984), which makes complete surgical removal almost impossible (Kaba and Kyritsis, 1997). Finding ways to attenuate glioma invasion is an important objective in glioma research. However, at present there are no effective treatments that attenuate glioma invasion.

While all glioma cells invade intraparenchymally, many cells follow nerve tracts or migrate along blood vessels (Zagzag et al., 2008) where a constant supply of oxygen and nutrients essential for their growth is assured. This positioning also exposes cells to a variety of factors including growth factors, chemokines, cytokines and kinins. The kinins are a family of signaling molecules of which bradykinin (BK) (Arg-Pro-Pro-Gly-Phe-Ser-Pro-Phe-Arg) (SEQ ID NO: 1) is the most prominent. BK is a product of high molecular weight kininogen (HMWK). Its cleavage is initiated by vascular endothelial cells where physiological activation of the kallikrein-kinin system leads to activation of prekallikrein to kallikrein (Moreau et al., 2005). Furthermore, astrocytes are capable of binding HMWK and low molecular weight kininogen on their surface and cleaving BK (Joseph and Kaplan, 2005). Kinins are normally present in the brain, but are upregulated under pathophysiological conditions that correlate with tumor progression/metastasis: hypoxia, tissue damage, and inflammation (Ratajczak et al., 2006). Of note, BK is an activator of matrix metalloproteinase (MMP) secretion (Hsieh et al., 2008), which may influence tissue remodeling (Ishiuchi et al., 2002).

Signals that attract glioma cells to blood vessels are poorly understood. It has been shown that vascular endothelial cells can initiate the BK signaling cascade and two BK receptors, B1 and B2 have been identified and cloned.

BK exerts many functions through binding to one of two BK receptors: bradykinin-1-receptor (B1R) expression is induced under pathological conditions while bradykinin-2-receptor (B2R) is constitutively active and responsible for physiological responses. BK receptors are G-protein coupled, and, after ligand binding, trigger a signal transduction cascade activating phospholipase β phosphoinositide breakdown, and PKC and calcium mobilization (Higashida et al., 2001).

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later. Nothing stated above should be interpreted as an admission that any reference meets the legal definition of “prior art.”

### SUMMARY

The following presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

It has been unexpectedly discovered that BK, acting via B2R, promotes migration of glioma cells. Low concentrations of BK stimulate sustained increases in intracellular Ca²⁺ concentration, whereas prolonged exposure to BK induces Ca²⁺ oscillations in glioma cells which, in turn, significantly enhance cell motility. In addition, invasion of glioma cells into brain slices and association with blood vessels are disrupted when B2R is pharmacologically inhibited or specific short-hairpin RNA (shRNA) constructs are used. This suggests that glioma cells use B2R to sense BK cleaved by endothelial cells and use this signal to identify and connect with blood vessels as they invade.

The disclosure provides novel targets for the treatment of cancer, such as glioma. The targets include BK and B2R. Methods are provided for modulating the migration of glial cells, including glioma cells. If the desired modulation is stimulation, the target may also be a molecule that stimulates (by increasing the expression, activity, or both) BK or B2R. If the desired modulation is inhibition (by decreasing the expression, activity, or both), the target may be a molecule that inhibits BK or B2R. The target may also be a downstream component of a biochemical pathway involving B2R.

The disclosure provides a composition for modulating cellular migration comprising a modulator that either stimulates or inhibits one of the targets listed above. Some embodiments of the modulator comprise at least one of an inhibitor of BK and an inhibitor of B2R. Such stimulation or inhibition may occur directly, or it may occur by stimulating or inhibiting one or more intermediate compounds in a biochemical pathway.

The disclosure provides a use of an inhibitor of one of the targets in the manufacture of a medicament for the treatment of cancer. In some embodiments the cancer is an invasive cancer, such as glioma.

The disclosure provides a method of modulating migration of a glial cell, said method comprising contacting the cell with a modulator of one of the targets. The modulator will either stimulate or inhibit the target.

The disclosure provides a pharmaceutical composition for treating cancer comprising a therapeutically effective amount of an inhibitor of a target. The disclosure provides methods of treating cancer comprising administering a therapeutically effective amount of the pharmaceutical composition to a subject in need thereof. The pharmaceutical composition and the method for treating cancer may be for the particular purpose of treating or preventing the migration of cancer cells (metastasis).

Methods are also provided for stimulating cancer or increasing metastasis in a subject (such as an animal model of cancer), comprising administering to the subject a compound that stimulates a target described above. The cancer may be brain cancer, or more specifically glioma. Methods are provided for stimulating glial migration in vitro, comprising contacting a cell with a compound that stimulates a target described above.

A method for diagnosing a subject at risk for cancer is provided, comprising measuring one or both of the activity and expression of a target in the subject and comparing it to a baseline level of activity and expression. A method is provided for determining the invasive potential of a cancer cell, said method comprising: measuring a property of the cell, said property selected from the group consisting of B2R activity and B2R expression; and comparing the property to a baseline value; wherein the cell is determined to have increased invasive potential if the property is higher than the baseline value. A kit is also provided for determining the invasive potential of a cancer cell in vitro, said kit comprising a means to measure the property of the cell.

Methods are provided for screening candidate compounds for the ability to modulate cellular migration. A general embodiment of the method comprises contacting the candidate compound to a ligand selected from B2R and BK; and determining binding between the candidate compound and the ligand; wherein the candidate compound is identified as a modulator of cellular migration if it is determined that the candidate binds to the ligand. Another general embodiment of the method comprises contacting the candidate compound to a ligand selected from B2R and BK; measuring the activity of the ligand; and comparing the activity of the ligand to a baseline value of the ligand's activity; wherein the candidate compound is identified as a modulator of cellular migration if the activity of the ligand differs from the baseline value. Another general embodiment of the method comprises contacting the candidate to a cell comprising B2R; measuring at least one property of B2R selected from the group consisting of B2R expression and B2R activity; and comparing the property to a baseline value for the property; wherein the candidate compound is identified as a modulator of cellular migration if the property differs from the baseline value.

A non-human animal model of invasive cancer is provided, comprising a cell that has been genetically altered to increase one or both of the expression and activity of one of the target molecules. Cell lines are provided for use in non-human animal models that comprise a genetically modified cell that over-expresses one of the target molecules or expresses a version of one of the target molecules with increased activity. Also provided is a non-human animal model that is resistant to invasive cancer, comprising a cell that has been genetically altered to decrease one or both of the expression and activity of one of the target molecules. Further provided is a non-human animal model of invasive cancer, comprising a cell that has been exposed to an activator of BK or B2K.

### BRIEF DESCRIPTION OF THE DRAWINGS

FIG. 1: B2R is expressed in glioma cell lines and patient tissue biopsies. A, normal human brain tissue and four GBM patient tissues (WHO I-IV) and D, glioma cell lines were stained with specific anti-B2R antibodies and counterstained with DAPI. Primary antibodies were omitted in controls (bottom panels in A and D); B, GBM patient tissue, WHO grade IV co-labeled with specific antibodies against B2R and laminin, and counterstained with DAPI; C, protein expression of B2R in non-nuclei protein enriched membrane fractions of glioma cell lines. The approximate molecular weight of the protein is indicated to the left as blot image was cropped for clarity. Scale bar=10 μm.

FIG. 2: Glioma cells respond to various stimuli by increasing intracellular calcium concentrations, yet only prolonged BK exposure results in calcium oscillations through binding to B2R. A, ATP, BK and ACh were applied on D54-MG cells previously loaded with FURA2-AM, and Ca²⁺-response was recorded; B, D54-MG loaded with FURA2-AM respond to various concentrations of BK by increase in intracellular Ca²⁺-concentrations; C, Representative example of prolonged exposure to BK resulting in frequent Ca²⁺-oscillations in cells (9/11); D, Last 5 min of a 30 min sequence time-lapse imaging of D54 cells loaded with Fura-2. Images were taken every 15 s; E, Calcium response to BK is abolished in the presence of specific B2R antagonists; Arrows in A, B, C and E indicate application of agonist. Arrows in D indicate a cell that changed position following calcium oscillations. Scale bar=10 μm.

FIG. 3: Glioma cell motility is increased in a BK concentration gradient. A, The first (A,a) and the last (A,b) frame of a time-lapse study of D54-MG-GFP cells exposed to a BK concentration gradient, increasing concentration from left to right; the group of cells in the white circle moved toward higher BK concentrations (black arrow indicates the overall distance change for that group of cells). Black star in a and b labels a random particle on the field of view ensuring that there was no shift of the chamber during the course of imaging. Frame/5 min over 5 h of imaging. Magnification 20×. B, A representative example of directionality for individual cell analysis reveals that in the presence of BK, 12/15 cells moved towards increasing BK concentration, red traces; C, Rose diagram presentation of averaged cell paths of D54-MG movement in an example from a representative field of view for each condition analyzed, indicating directionality was affected in BK concentration gradient while in the control (8/15) and in the presence of HOE-140 (8/15) or Bradyzide (6/15) cells were moving in random fashion; Da, presence of BK concentration gradient significantly increased velocity and Db, distance traveled by D54-MG glioma cells. All data reported are mean±S.E.M. Statistical analysis, One-way ANOVA (*<0.05, ** p<0.01).

FIG. 4: Transwell glioma cell migration/invasion assays suggest that BK enhances invasive migration of glioma cells. Aa, Analysis of migration assay, effects of ATP, ACh and BK: BK significantly increases migration; Ab, Representative fluorescent images of D54-MG-GFP fixed after 5 hr of migration and counterstained with DAPI; Ba, Analysis of migration assay, effects of different BK concentrations in presence or absence of B2R antagonists: BK significantly increases migratory properties of glioma cells in concentration dependant manner, the effect was abolished in the presence of antagonists; Bb, Representative fluorescent images of D54-MG-GFP migrated for 5 hr in 0, 0.1, 0.3 or 1 μM BK. After fixation, cells were counterstained with DAPI; C, Mean data from the invasion assay suggest that BK increased glioma cells significantly at 0.3 μM BK concentration; the effect was abolished in the presence of antagonists; D,a, Analysis of fluorescent intensities; b, Representative fluorescent images of D5-MG after 24 h incubation in gelatin DQ and BK in presence or absence of B2R antagonists. After fixation, cells were counterstained with DAPI. Columns, percent control, bars, S.E.M. Statistical analysis, One-way ANOVA (* p<0.05). Scale bar=10 μm.

FIG. 5: BK enhances cell invasion in brain slices. A, Representative immunofluorescent images of D54-MG-GFP cells fixed 2 h after brain slices invasion. D54-MG-GFP (green) cells enwrapping blood vessels (red) in the presence of BK (Aa) while invading brain slice. Addition of B2R antagonist dramatically reduces the number of cells on the blood vessels (Ab,c), as quantified in B; Cross-section of cells in reconstructed z-stack indicate deeper penetration of glioma cells enwrapping blood vessel, 50 μm (Ad) with addition of BK than in presence of B2R antagonist (Ae), 27 μm imaging plane, or only B2R antagonist, 40 μm imaging plane (Af). C, Experimental set-up for slice invasion assay. D, Percentage of the D54-MG-GFP cells at certain depth as they migrate/invade from the top of the slice, presence of BK causes shift to the right/deeper into the tissue (red) compared to controls (black) or to addition of B2R antagonist when most of the cells remain on the top of the slice (green, blue). Columns, percent control, bars, S.E.M. Statistical analysis, One-way ANOVA (* p<0.05). Scale bar=10 μm.

FIG. 6: BK effects on glioma cells are due to actions of B2R. A, Representative Western blot displaying reduced B2R protein expression in shRNA transfected cells when doxycycline treated for 5 days. The approximate molecular weight of the protein is indicated to the left as blot image was cropped for clarity; B a, b and c, Control (scramble) and non-induced D54-MG-GFP cells show normal Ca²⁺-response after BK stimulation (as indicated by the arrow), while doxycycline induced B2R knockdowns failed to respond to the stimulation; C, Migration assay analysis of doxycycline treated shRNA transfected cells in the presence or absence of BK: migration of control (scramble) cells was significantly increased in the presence of BK while migration of B2R knockdowns was insensitive to BK and significantly decreased compared to control (scramble) cells; D, Slice invasion assay was performed with doxycycline treated cells: the percentage of control cells (scramble) on blood vessels is significantly increased with addition of BK while in case of shRNA transfected D54-MG-GFP, BK did not affect attachment onto the blood vessel, and is significantly reduced compared to controls. Columns, percent control, bars, S.E.M. Statistical analysis, Student t-test (* p<0.05) and One-way ANOVA (** p<0.01, *** p<0.001).

FIG. 7: Tumor invasion of living mouse brain slices in situ. The upper images show the spread of fluorescent tumor cells in a slice exposed to BK (control) and a slice exposed to BK and HOE-140. The lower bar graphs show percent change in tumor size (bars are the standard error of the mean).

FIG. 8: Homology alignment of B2R in several species showing conserved regions.

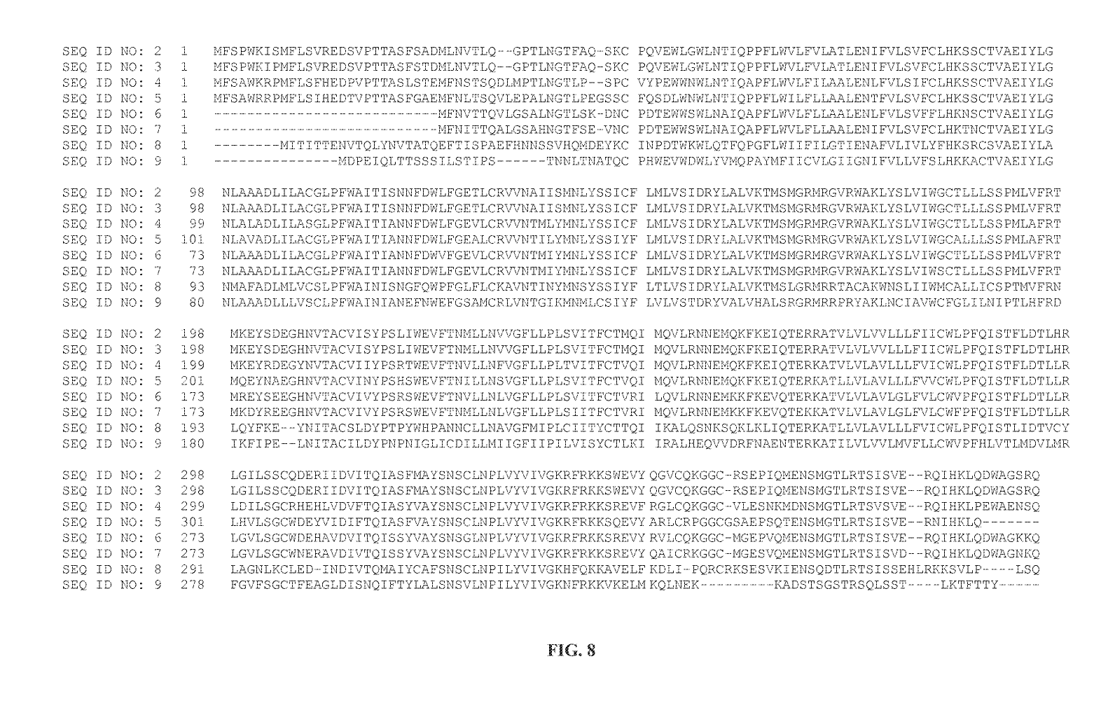

FIG. 9: Co-localization of B2R and GFAP in patient tissue biopsies. Normal human brain tissue and four GBM patient tissues (WHO I-IV) were stained with specific anti-B2R and GFAP antibodies and counterstained with DAPI. Scale bar 10 μm.

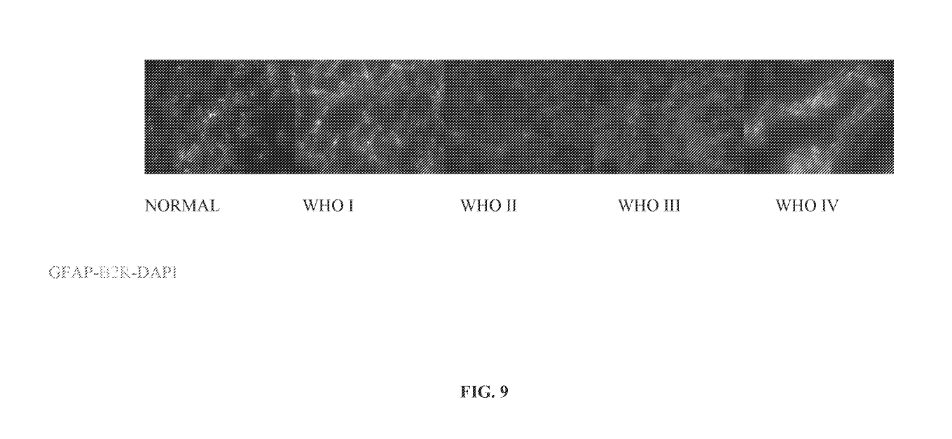

FIG. 10: Expression of bradykinin receptors on glioma cells. Immunostaining of D54-MG treated with specific antibodies against B1R (A) and B2R (B). C, Immunoreactivity was completely abolished when primary antibodies were omitted. D, Fluorescent intensities, expressed in intensity units (i.u.) were quantified and compared to control. Expression of B2R is significantly stronger when compared with control where primary antibody was omitted or with BIR staining, while BIR signal does not significantly differ from the control. All data reported are mean±S.E.M. Statistical analysis, One-way ANOVA (** p<0.01). Scale bar=10 μm.

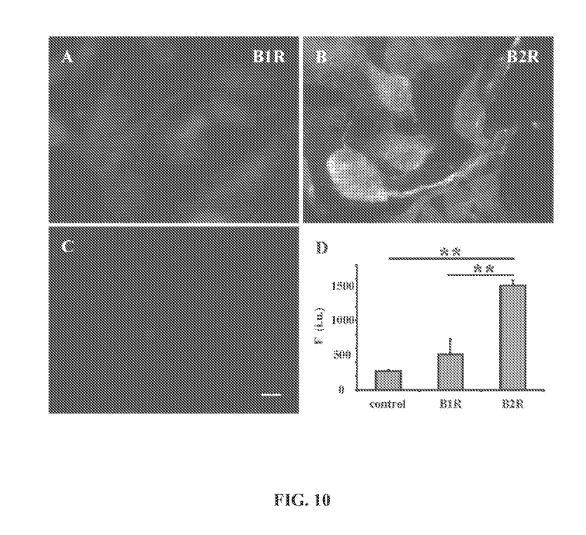

FIG. 11: Analysis of D54 individually tracked cells in a bradykinin concentration gradient. A, Average velocities of D54 cells in control experiments was 0.52±0.05 μm/min with a significant increase to 0.68±0.05 μm/min in the BK gradient. Statistical analysis, Oneway ANOVA (** p<0.01); B, Increase in average distances was from 157.6±8.0 in control experiment to 203.0±8.1. All data reported are mean±S.E.M.

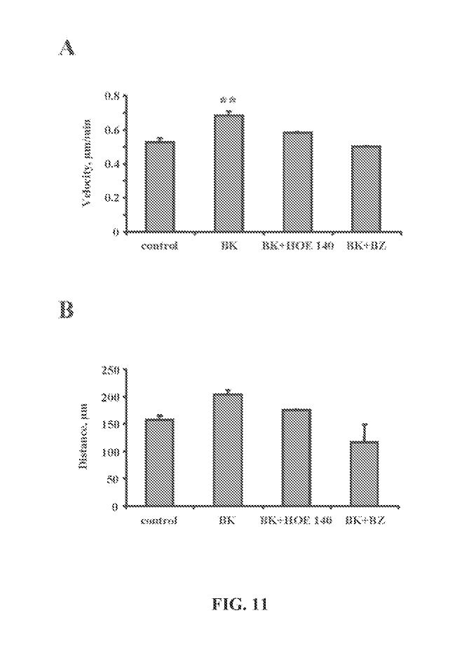

FIG. 12: Analysis of D54 individually tracked cells in a bradykinin concentration gradient. A, A, Average velocities of U251 cells in control experiments was 0.26±0.02 μm/min with a significant increase to 0.46±0.07 μm/min in the BK gradient; B, Increase in average distances was from 77.8±5.3 in control experiment to 136.0±12.8. All data reported are mean±S.E.M.

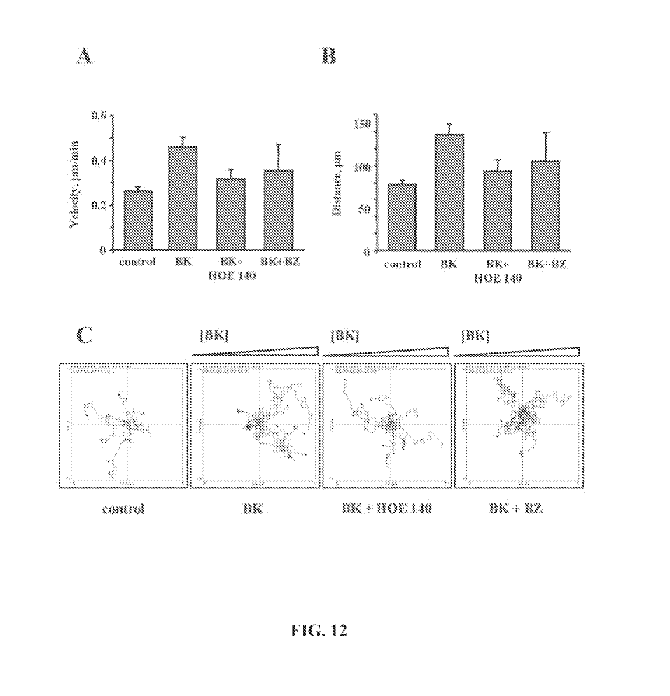

FIG. 13: Representative immunofluorescent images of D54-MG-GFP cells fixed 2 h after brain slice invasion. D54-MG-GFP (green) cells enwrapping blood vessels (red) in the presence of BK while invading brain slice. Most cells remained on the top of slices and not associating with blood vessels after addition of B2R antagonist. Scale bar=100 μm.

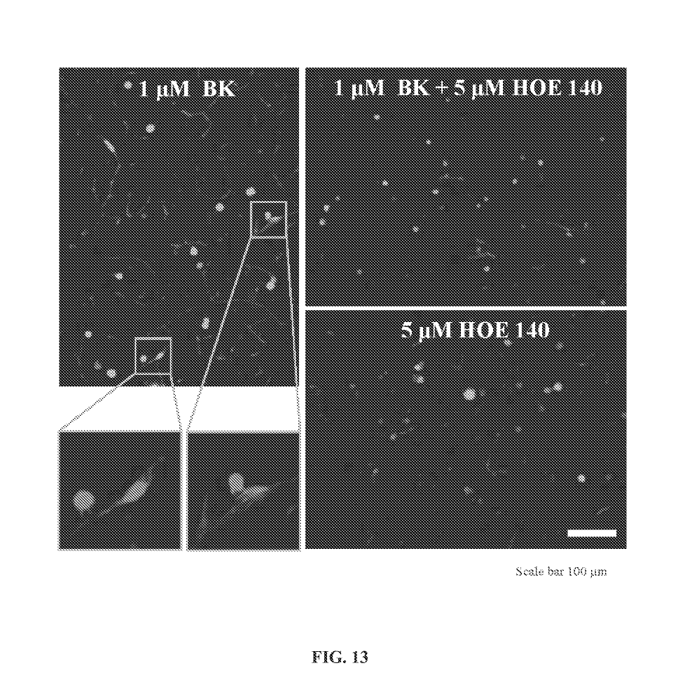

FIG. 14: Representative confocal images of both glioma cell lines stably expressing shRNA to suppress the expression of B2R. Cells were fixed after 2 h of invasion into slices in the presence of BK. The bottom panels show cross-sections of reconstructed z-stacks indicating most of the cells remained on the top of the slice and not associated with blood vessels, 30 μm sections. Scale bar=10 μm.

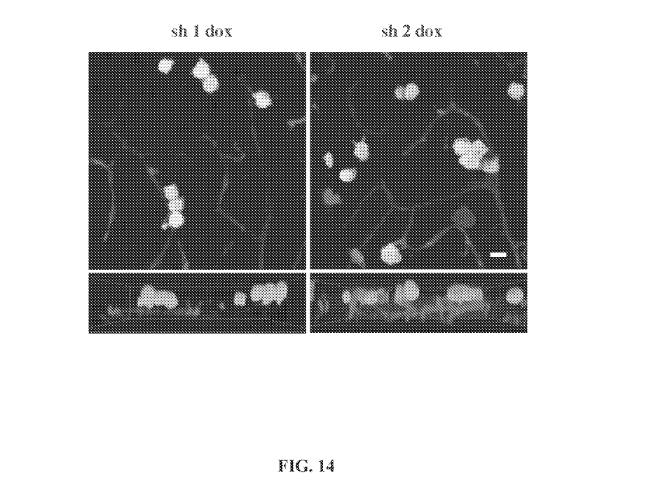

### DETAILED DESCRIPTION

This disclosure provides BK and B2R targets for cell migration. Without wishing to be bound by any hypothetical model, BK appears to act on cell migration through its interaction with B2R. B2R is a cell surface receptor. Its existence has been confirmed through the use of high affinity peptide and non-peptide receptor antagonists, radioligand binding studies and, recently, receptor cloning and expression studies (Hall, 1992). Molecular cloning techniques have identified the gene encoding B2R receptors in various species. The bradykinin-1-receptor and B2R show little (36%) overall sequence homology. Cloning studies reveal the potential for the existence of species homologues of receptors. The two classification criteria, namely the order of potency of agonists and the actual affinity of antagonists have been found to be applicable for receptor classification based not on data only from bioassays but also from other approaches (binding assays, molecular biology techniques). The preferred agonist for the B2 receptor, which accounts for the majority of the acute pharmacological effects of bradykinin, is bradykinin itself. Kallidin also acts on the B2 receptor but in addition can act here following conversion to bradykinin by the action of aminopeptidases (Couture, 2001).

The gene for B2R has been cloned and the receptor sequenced (McEachern 1991; Hess 1992). Human variant, UniProtKB/Swiss-Prot: P30411.2, contains 391 amino acids, a disulfide bond, three glycosylation sites and one lipid-binding site (source: NCBI). B2R receptor belongs to the family of receptors with 7-trans membrane spanning domains and is G-protein-coupled (Gα and Gq) to a number of biochemical pathways via a system of second messengers: phospholipase C induces the formation of IP3 and DAG which mobilizes intracellular calcium and activates protein kinase C (PKC) respectively; arachidonic acid can be generated from membrane phospholipids via phospholipase A2 activity and from DAG. Following increase in intracellular calcium concentration, the formation of cAMP, cGMP and nitric oxide (NO) is stimulated (Couture, 2001).

### DEFINITIONS

The terms “prevention”, “prevent”, “preventing”, “suppression”, “suppress” and “suppressing” as used herein refer to a course of action (such as administering a compound or pharmaceutical composition of the present disclosure) initiated prior to the onset of a clinical manifestation of a disease state or condition so as to prevent or reduce such clinical manifestation of the disease state or condition. Such preventing and suppressing need not be absolute to be useful.

The terms “treatment”, “treat” and “treating” as used herein refers a course of action (such as administering a compound or pharmaceutical composition) initiated after the onset of a clinical manifestation of a disease state or condition so as to eliminate or reduce such clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method, compound or pharmaceutical composition of the disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method, compound or pharmaceutical composition of the disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

The term “therapeutically effective amount” as used herein refers to an amount of a substance, either alone or as a part of a pharmaceutical composition, that is capable of having any detectable, positive effect on any symptom, aspect, or characteristics of a disease state or condition. Such effect need not be absolute to be beneficial.

The term “prodrug” as used herein includes functional derivatives of a disclosed compound which are readily convertible in vive into the required compound. Thus, in the methods of treatment of the present disclosure, the term “administering” shall encompass the treatment of the various disease states/conditions described with the compound specifically disclosed or with a prodrug which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The term “pharmaceutically acceptable salts” as used herein includes salts of the active agents which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

### Inhibitors

The present disclosure provides for inhibitors that inhibit at least one of BK activity and B2R activity, either directly or through inhibition of expression, either in vitro or in vivo. For some inhibitors the inhibition may occur through direct interaction between the inhibitor and BK or B2R. For some inhibitors the inhibition may occur indirectly, through the modulation of an intermediary compound. Some embodiments of the inhibitor act by inhibiting polypeptide regulated by B2R and/or BK. For the purposes of this section, the term “inhibition target” refers to one of BK, B2R, a polypeptide regulated by BK, and a polypeptide regulated by B2R. Of course, an inhibitor could potentially function to inhibit two or more of the targets.

Inhibiting the activity of a polypeptide regulated by at least one of BK and B2R as used herein refers to modulating the function of such polypeptide in a manner opposite of is regulation by at least one of BK and B2R. For example, if at least one of BK and B2R stimulates the activity or induces translocation a given polypeptide, then modulating the activity of such polypeptide refers to inhibiting the activity of such polypeptide or inhibiting translocation. Likewise, if at least one of BK and B2R inhibits the activity or inhibits translocation of a given polypeptide, then modulating the activity of such polypeptide refers to stimulating the activity of such polypeptide or inducing translocation.

Such inhibitors can exert their effect on the activity of the inhibition target via changes in expression, post-translational modifications or by other means. Suitable inhibitors include, but are not limited to, polypeptides, functional nucleic acids, carbohydrates, antibodies, small molecules, or any other molecule which decrease the activity of the inhibition target. Such inhibitors may be identified in the methods of screening discussed herein.

In certain embodiments, the inhibitor does not inhibit the bradykinin-1-receptor, directly or indirectly.

### Small Molecules and Peptidomemetic Compounds

In one embodiment, the inhibitors of the present disclosure are small molecules or peptidomemetic compounds. In further embodiments the inhibitor is HOE-140, Icatibant, bradyzide, a derivative of one of the foregoing, a tautomer of any of the foregoing, and a salt of any of the foregoing.

In a specific embodiment, the small molecule is bradyzide, a derivative of bradyzide, or a tautomer of bradyzide. Bradyzide is from class of rodent-selective non-peptide B2 BK antagonists (1-(2-Nitrophenyl)thiosemicarbazides) (Burgess et al., British J. Pharm. (2000) 129:77-86—incorporated herein by reference to teach the use of this compound). Bradyzide has high affinity for the B2R, having been observed to displace [³H]-BK binding in NG108-15 cells and in Cos-7 cells expressing the rat receptor with KI values of 0.51+0.18 nM (n=3) and 0.89+0.27 nM (n=3), respectively. Bradyzide is a competitive antagonist, having been observed to inhibit B2 receptor-induced ⁴⁵Ca efflux from NG108-15 cells with a pK_{B} of 8.0±0.16 (n=5) and a Schild slope of 1.05. In the rat spinal cord and tail preparation, bradyzide inhibits BK-induced ventral root depolarizations (IC₅₀ value; 1.6±0.05 nM (n=3)). Bradyzide inhibits BK-induced [³H]-inositol trisphosphate (IP3) formation with IC₅₀ values of 11.6±1.4 nM (n=3) at the rat and 2.4±0.3 mM (n=3) at the human receptor. Bradyzide does not interact with a range of other receptors, including human and rat B1 BK receptors. Bradyzide is orally available and blocks BK-induced hypotension and plasma extravasation. In summary, bradyzide is a potent, orally active, antagonist of B2R, with selectivity for the rodent over the human receptor. Bradyzide has the following structure:

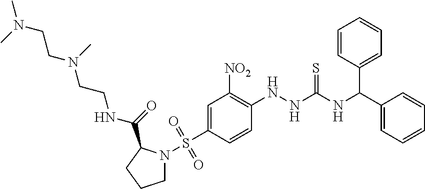 [SMILES: CN(C)CCN(C)CCNC(=O)[C@@H]1CCCN1S(=O)(=O)c1ccc(NNC(=S)NC(c2ccccc2)c2ccccc2)c([N+](=O)[O-])c1]

In another specific embodiment, the small molecule is HOE-140 or a peptide comprising HOE-140. HOE-140 has the structure (D-Arg-[Hyp³, Thi⁵, D-Tic⁷, Oic⁸]bradykinin; D-Arg-L-Arg-L-Pro-L-Hyp-Gly-L-(2-thienyl)Ala-L-Ser-D-1,2,3,4-tetrahydro-3-isoquinolinecarbonyl-L-(2α,3β,7aβ)-octahydro-1H-indole-2-carbonyl-L-Arg; H2N-D-Arg-Arg-Pro-Hyp-Gly-Thi-Ser-D-Tic-Oic-Arg; CAS No. 138614-30-9) and is a (BK)-antagonist (Hock et al., Br. J. Pharmacol. (1991):102, 769-773—incorporated herein by reference to teach the use of this compound). In receptor binding studies in guinea-pig ileum preparations, HOE-140 displays an IC₅₀ of 1.07×10⁹ mol L⁻¹ and a K₁ value of 7.98×10⁻¹⁰ mol⁻¹. HOE-140 displays two to three orders of magnitude more potency than D-Arg-[Hyp², Thi^{5,8}, D-Phe⁷]BK. The structure of HOE-140 is shown below:

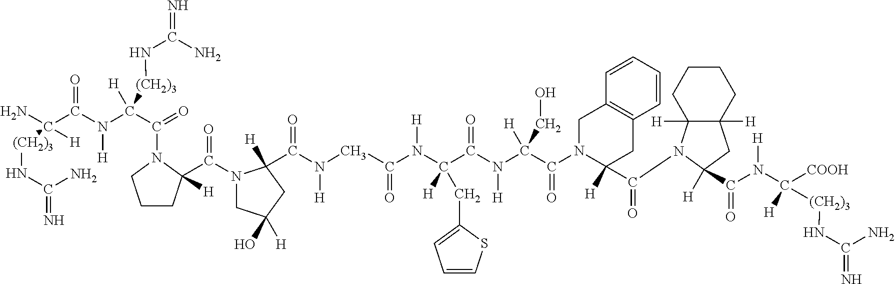

Icatibant is a peptidomemetic compound comprising ten amino acid residues ((2s)-2-({[(3as,7as)-1-({2-[(2s)-2-{[(2s)-2-({[({(4r)-1-[(1-{2-({(2r)-2-amino-5-[(diaminomethylidene)amino]pentanoyl}amino)-5-[(diaminomethylidene)amino]pentanoyl}pyrrolidin-2-yl)carbonyl]-4-hydroxypyrrolidin-2-yl}carbonyl)amino]acetyl}amino)-3-(thiophen-2-yl)propanoyl]amino}-3-hydroxypropanoyl]-1,2,3,4-tetrahydroisoquinolin-3-yl}carbonyl)octahydro-1h-indol-2-yl]carbonyl}amino)-5-[(diaminomethylidene)amino]pentanoic acid; CAS number 130308-48-4). The structure of icatibant is shown below:

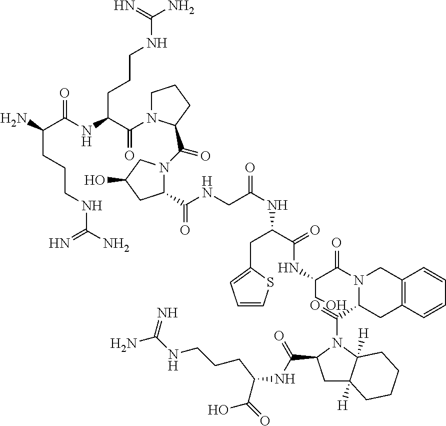 [SMILES: N=C(N)NCCC[C@@H](N)C(=O)N[C@@H](CCCNC(=N)N)C(=O)N1CCC[C@H]1C(=O)N1C[C@H](O)C[C@H]1C(=O)NCC(=O)N[C@@H](Cc1cccs1)C(=O)N[C@@H](CO)C(=O)N1Cc2ccccc2C[C@@H]1C(=O)N1[C@H](C(=O)N[C@@H](CCCNC(=N)N)C(=O)O)C[C@@H]2CCCC[C@@H]21]

Icatibant is a potent antagonist of B2R (Bork, Nature Reviews Drug Discovery 7, 801-802 (October 2008)—incorporated by reference herein only to teach the identity and use of this compound as a B2R inhibitor).

In further embodiments the small molecule is the phosphonium-derived WIN 64338 and the heteroaryl benzyl ethers FR173657 and FR193517 (Salvino et al., 1993; Asano et al., 1997; Abe et al., 1998—all of which are incorporated herein by reference to teach the use of these compounds as B2R inhibitors).

### Nucleic Acid Inhibitors

In one embodiment, the inhibitors of the present disclosure are functional nucleic acids. Functional nucleic acids are nucleic acid molecules that carry out a specific function in a cell, such as binding a target molecule or catalyzing a specific reaction.

Such functional nucleic acids may inhibit the activity of an inhibition target (nucleic acid inhibitors). Functional nucleic acids include but are not limited to antisense molecules, aptamers, ribozymes, triplex forming molecules, small interfering RNA (siRNA), RNA interference (RNAi), and external guide sequences (EGS). In one embodiment, a siRNA could be used to reduce or eliminate expression of at least one inhibition target.

Antisense molecules are designed to interact with a target nucleic acid molecule through either canonical or non-canonical base pairing. The interaction of the antisense molecule and the target molecule is designed to promote the destruction of the inhibition target through, for example, RNAseH mediated RNA-DNA hybrid degradation.

Alternatively, the antisense molecule is designed to interrupt a processing function that normally would take place on the target nucleic acid molecule, such as transcription or replication. Antisense molecules can be designed based on the sequence of the target nucleic acid molecule (such as a nucleic acid encoding an inhibition target). Numerous methods for optimization of antisense efficiency by finding the most accessible regions of the target nucleic acid molecule exist. Exemplary methods include, but are not limited to, in vitro selection experiments and DNA modification studies using DMS and DEPC.

Aptamers are molecules that interact with a target nucleic acid molecule, often in a specific way. Typically aptamers are small nucleic acids ranging from 15-50 bases in length that fold into defined secondary and tertiary structures, such as stem-loops or G-quartets. Representative examples of how to make and use aptamers to bind a variety of different target nucleic acid molecules can be found in, for example, U.S. Pat. Nos. 5,476,766 and 6,051,698 (which are hereby incorporated by reference for this teaching). The secondary structure inhibits expression of the polypeptide encoded by the gene or inhibits a processing function as discussed above.

Ribozymes are nucleic acid molecules that are capable of catalyzing a chemical reaction, either intramolecularly or intermolecularly. There are a number of different types of ribozymes that catalyze nuclease or nucleic acid polymerase type reactions which are based on ribozymes found in natural systems, such as, but not limited to, hammerhead ribozymes, hairpin ribozymes and tetrahymena ribozymes. There are also a number of ribozymes that are not found in natural systems, but which have been engineered to catalyze specific reactions de novo (including, but not limited to, those described in U.S. Pat. Nos. 5,807,718, and 5,910,408, which are hereby incorporated by reference for this teaching). Ribozymes may cleave RNA or DNA substrates. Representative examples of how to make and use ribozymes to catalyze a variety of different reactions can be found in U.S. Pat. Nos. 5,837,855; 5,877,022; 5,972,704; 5,989,906; and 6,017,756 (which are hereby incorporated by reference for this teaching).

Triplex forming functional nucleic acid molecules are nucleic acid molecules that can interact with either double-stranded or single-stranded nucleic acid. When triplex forming nucleic acids interact with a target region, a structure called a triplex is formed, in which three strands of DNA form a complex dependant on both Watson-Crick and Hoogsteen base-pairing. Triplex molecules can bind target regions with high affinity and specificity. Representative examples of how to make and use triplex forming molecules to bind a variety of different target nucleic acid molecules can be found in U.S. Pat. Nos. 5,650,316; 5,683,874; 5,693,773; 5,834,185; 5,869,246; 5,874,566; and 5,962,426 (which are hereby incorporated by reference for this teaching).

EGSs are molecules that bind a target nucleic acid molecule forming a complex, which is recognized by RNase P. RNaseP then cleaves the target nucleic acid molecule. EGSs can be designed to specifically target a RNA molecule of choice. Representative examples of how to make and use EGS molecules to facilitate cleavage of a variety of different target nucleic acid molecules may be found in U.S. Pat. Nos. 5,168,053; 5,624,824; 5,683,873; 5,728,521; 5,869,248; and 5,877,162 (which are hereby incorporated by reference for this teaching).

Gene expression can also be effectively silenced in a highly specific manner through RNA interference (“RNAi”). Small interfering RNA (“siRNA”) is a double-stranded RNA that can induce sequence-specific post-transcriptional gene silencing, thereby decreasing or even inhibiting gene expression from a target nucleic acid. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer, siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. siRNA can also be synthesized in vitro using kits such as Ambion's SILENCER®, siRNA Construction Kit (Ambion, Austin, Tex.).

### Antibody Inhibitors

Polypeptides that inhibit at least one of the inhibition targets include antibodies with antagonistic or inhibitory properties. In addition to intact immunoglobulin molecules, fragments, chimeras, or polymers of immunoglobulin molecules are also useful in the methods taught herein, as long as they are chosen for their ability to inhibit at least one of the inhibition targets. The antibodies can be tested for their desired activity using in vitro assays, or by analogous methods, after which their in vivo therapeutic or prophylactic activities are tested according to known clinical testing methods.

The term “antibody” is used herein in a broad sense and includes both polyclonal and monoclonal antibodies. Monoclonal antibodies can be made using any known procedure. For example, disclosed monoclonal antibodies can be prepared using hybridoma methods, such as those described by Kohler and Milstein, Nature, 256:495 (1975) (which is incorporated by reference herein for this teaching). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. The monoclonal antibodies may also be made by recombinant DNA methods, such as those described in U.S. Pat. No. 4,816,567 (which is hereby incorporated by reference for this teaching). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, as described in U.S. Pat. No. 5,804,440 and U.S. Pat. No. 6,096,441 (which are hereby incorporated by reference for this teaching).

Antibody fragments include Fv, Fab, Fab′ or other antigen binding portion of an antibody. Digestion of antibodies to produce fragments thereof can be accomplished using routine techniques known in the art. For instance, digestion can be performed using a protease, such as papain. Examples of papain digestion are described in WO 94/29348 published and U.S. Pat. No. 4,342,566 (which are hereby incorporated by reference for this teaching). Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antibodies or antibody fragments may also include insertions, deletions, substitutions, or other selected modifications of particular regions or specific amino acids residues. These modifications can provide additional or improved function. For example, the removal or addition of acids capable of disulfide bonding may increase the bio-longevity of the antibody. In any case, the modified antibody or antibody fragment retains a desired bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment (Zoller, M. J. Curr. Opin. Biotechnol. 3:348-354, 1992).

The antibody or antibody fragment can be a mammalian antibody or an avian antibody. The antibody may be a human antibody or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86 95, 1991). Human antibodies (and fragments thereof) can also be produced using phage display libraries (Hoogenboom et al., J. Mol. Biol., 227:381, 1991; Marks et al., J. Mol. Biol., 222:581, 1991). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551 255 (1993); Jakobovits et al., Nature, 362:255 258 (1993); Bruggermann et al., Year in Immunol., 7:33 (1993)).

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. Methods for humanizing non human antibodies are well known in the art. For example, humanized antibodies can be generated according to the methods of Winter and coworkers (Jones et al., Nature, 321:522 525 (1986), Riechmann et al., Nature, 332:323 327 (1988), Verhoeyen et al., Science, 239:1534 1536 (1988)), by substituting rodent CDRs or CDR sequences for the corresponding sequences of a human antibody. Methods that can be used to produce humanized antibodies are also described in U.S. Pat. Nos. 4,816,567, 5,565,332, 5,721,367, 5,837,243, 5, 939,598, 6,130,364, and 6,180,377.

### Compositions

Compositions are provided comprising a modulator of at least one of BK, B2R, or a compound regulated by either. Some embodiments of the compositions comprise one or more compounds useful in the treatment and prevention methods of the present disclosure, such as, but not limited to, those inhibitors described above. In one embodiment, such compounds decrease the expression, in whole or in part, of the at least one of the BK and B2R gene, thereby reducing the levels of such proteins in the subject. In an alternate embodiment, such compounds decrease the activity, in whole or in part, of at least one of BK and B2R, so as to reduce the activity/activation of the at least one of BK and B2R receptor and/or downstream signaling pathways of the at least one of BK and B2R receptor in the subject. In yet another alternate embodiment, such compounds decrease the activity, number or distribution, in whole or in part, of resident non-hematopoietic cells expressing at least one of BK and B2R, thereby decreasing the activation of these cells in the presence of endogenous activators of at least one of BK and B2R receptor. For the purposes of the below discussion, “active agents” means any such modulators, including the inhibitors described above. Some embodiments of the composition may comprise more than a single active agent.

Some embodiments of the compositions are pharmaceutical compositions. The compositions disclosed may comprise one or more active agents, in combination with a pharmaceutically acceptable carrier. Examples of such carriers and methods of formulation may be found in Remington: The Science and Practice of Pharmacy (20^{th} Ed., Lippincott, Williams & Wilkins, Daniel Limmer, editor). To form a pharmaceutically acceptable composition suitable for administration, such compositions will contain a therapeutically effective amount of an active agent.

The pharmaceutical compositions of the disclosure may be used in the treatment and prevention methods of the present disclosure. Such compositions are administered to a subject in amounts sufficient to deliver a therapeutically effective amount of the active agent(s) so as to be effective in the treatment and prevention methods disclosed herein. The therapeutically effective amount may vary according to a variety of factors such as, but not limited to, the subject's condition, weight, sex and age. Other factors include the mode and site of administration. The pharmaceutical compositions may be provided to the subject by any method known in the art. Exemplary routes of administration include, but are not limited to, subcutaneous, intravenous, topical, epicutaneous, oral, intraosseous, intramuscular, intranasal and pulmonary. The active agents of the present disclosure may be administered only once to the subject, or more than once to the subject. Furthermore, when the compositions are administered to the subject more than once, a variety of regimens may be used, such as, but not limited to, one per day, once per week, once per month or once per year. The compositions may also be administered to the subject more than one time per day. The therapeutically effective amount of the active agent and appropriate dosing regimens may be identified by routine testing in order to obtain optimal activity, while minimizing any potential side effects. In addition, co-administration or sequential administration of other agents may be desirable.

The compositions of the present disclosure may be administered systemically, such as by intravenous administration, or locally such as by subcutaneous injection or by application of a paste or cream.

Some embodiments of the pharmaceutical composition are formulated to facilitate delivery of the active agent to a certain tissue, organ, or system. A specific embodiment of the pharmaceutical composition is formulated to facilitate delivery of the active agent to neural tissue. A further specific embodiment of the pharmaceutical composition is formulated to facilitate delivery of the active agent to the nervous system; in more particular embodiments the pharmaceutical composition is formulated to facilitate delivery of the active agent to one of the central nervous system or the peripheral nervous system.

Further embodiment of the pharmaceutical composition are formulated to facilitate the delivery of the active agent to a cancer cell or to a tumor. This can be accomplished by various means known in the art. In some embodiments of the composition such delivery is facilitated using a selective ligand that has a stronger tendency to bind to cancer cells than to non-cancer cells. In some embodiments of the composition such delivery is facilitated using an antibody or an antibody fragment. Such delivery means may be conjugated to the active agent. Alternatively, such delivery means may be incorporated into a delivery vehicle, such as a liposome.

In a further specific embodiment the pharmaceutical composition is formulated to facilitate delivery of the active agent to the brain. Such formulation may increase the rate at which the active agent crosses the blood-brain barrier. Such formulation may also render the composition suitable for intrathecal or intraventricular administration.

The compositions of the present disclosure may further comprise agents which improve the solubility, half-life, absorption, or other characteristics of the active agent. Furthermore, the compositions of the present disclosure may further comprise agents that attenuate undesirable side effects and/or or decrease the toxicity of the active agent. Examples of such agents are described in a variety of texts, such as, but not limited to, Remington: The Science and Practice of Pharmacy (20^{th} Ed., Lippincott, Williams & Wilkins, Daniel Limmer, editor).

The compositions of the present disclosure can be administered in a wide variety of dosage forms for administration. For example, the compositions can be administered in forms, such as, but not limited to, tablets, capsules, sachets, lozenges, troches, pills, powders, granules, elixirs, tinctures, solutions, suspensions, elixirs, syrups, ointments, creams, pastes, emulsions, or solutions for intravenous administration, intrathecal administration, intraventricular, administration, or injection. Other dosage forms include administration transdermally, via patch mechanism or ointment. Further dosage forms include formulations suitable for delivery by nebulizers or metered dose inhalers. Any of the foregoing may be modified to provide for timed release and/or sustained release formulations.

In the present disclosure, the pharmaceutical compositions may further comprise a pharmaceutically acceptable carrier. Such carriers include, but are not limited to, vehicles, adjuvants, surfactants, suspending agents, emulsifying agents, inert fillers, diluents, excipients, wetting agents, binders, lubricants, buffering agents, disintegrating agents and carriers, as well as accessory agents, such as, but not limited to, coloring agents and flavoring agents (collectively referred to herein as a carrier). Typically, the pharmaceutically acceptable carrier is chemically inert to the active agents and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, for oral administration in solid form, such as but not limited to, tablets, capsules, sachets, lozenges, troches, pills, powders, or granules, the active agent may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid as well as the other carriers described herein. Lozenge forms can comprise the active agent in a flavor, usually sucrose and acacia or tragacanth, as well as pastilles comprising the active ingredient in an inert base, such as gelatin and glycerin, or sucrose and acadia, emulsions, and gels containing, in addition to the active agent, such carriers as are known in the art.

For oral liquid forms, such as but not limited to, tinctures, solutions, suspensions, elixirs, syrups, the active agent of the present disclosure can be dissolved in diluents, such as water, saline, or alcohols. Furthermore, the oral liquid forms may comprise suitably flavored suspending or dispersing agents such as the synthetic and natural gums, for example, tragacanth, acacia, methylcellulose and the like. Moreover, when desired or necessary, suitable and coloring agents or other accessory agents can also be incorporated into the mixture. Other dispersing agents that may be employed include glycerin and the like.

Formulations suitable for parenteral administration include aqueous and non-aqueous, isotonic sterile injection solutions, which can contain anti-oxidants, buffers, bacteriostats, and solutes that render the formulation isotonic with the blood of the patient (or isotonic with another bodily fluid into which the composition will be administered, such as cerebrospinal fluid), and aqueous and non-aqueous sterile suspensions that can include suspending agents, solubilizers, thickening agents, stabilizers, and preservatives. The active agent may be administered in a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as, but not limited to, a soap, an oil or a detergent, suspending agent, such as, but not limited to, pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkylbeta-aminopropionates, and 2-alkylimidazoline quaternary ammonium salts, and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17.

Topical dosage forms, such as, but not limited to, ointments, creams, pastes, emulsions, containing the nucleic acid molecule of the present disclosure, can be admixed with a variety of carrier materials well known in the art, such as, e.g., alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage or dressing for transdermal delivery.

The active agent of the present disclosure can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. Such liposomes may also contain monoclonal antibodies to direct delivery of the liposome to a particular cell type or group of cell types.

The active agent of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

### Methods of Treatment and Prevention

The teachings of the present disclosure provide a method for the treatment and/or prevention of disease states and conditions associated with or characterized by cancer in a subject in need of such treatment. Such disease states and conditions include, but are not limited to, brain cancer, invasive cancer, and glioma.

The method of treatment and/or prevention comprises administering to the subject any of the pharmaceutical compositions disclosed herein. The method will often further comprise identifying a subject in need of such treatment or prevention.

In one embodiment, the treatment and/or prevention is accomplished by decreasing the expression, in whole or in part, of the at least one of the BK and B2R gene, to reduce the levels of such polypeptides in the subject. Such decreased expression is accomplished by administering a pharmaceutical composition containing at least one agent capable of decreasing the expression of such genes, such as a functional nucleic acid which may be delivered via gene-therapy or other techniques known in the art.

In an alternate embodiment, the treatment and/or prevention is accomplished by decreasing the activity, in whole or in part, of at least one of BK, B2R, and a polypeptide regulated by the at least one of the foregoing. Such decreased activity is accomplished by administering a pharmaceutical composition containing at least one active agent such as but not limited to, a specific or non-specific inhibitor of such polypeptides, agents that reduce the stability or half-life of the such polypeptides, or agents that promote the intracellular sequestration of the such polypeptides.

In yet another embodiment, said treatment and/or prevention is accomplished by decreasing the activity, number or distribution, in whole or in part, of cells expressing at least one of 3BK and B2R, thereby decreasing the activation of these cells in the presence of endogenous activators of at least one of BK and B2R. Such decreased activation is accomplished by administering an agent capable of decreasing the expression of at least one of BK and B2R or decreasing the activation of such cells, such as but not limited to, factors that decrease the activation, number or distribution of cells expressing at least one of BK and B2R, agents, such as, but not limited to, antibodies that sequester factors that activate cells expressing at least one of BK and B2R, increasing the expression of factors that decrease the activation of cells expressing at least one of BK and B2R or decreasing the gene expression of factors that activated cells expressing at least one of BK and B2R. Such modulation would thereby reduce at least one of BK and B2R mediated activation of cells expressing at least one of BK and B2R receptor in a subject and treat the disease states or conditions discussed herein.

In certain embodiments of the treatment and/or prevention methods, the results of inhibiting the activity and/or expression of at least one of BK and B2R or a polypeptide regulated by at least one of BK and B2R include, but are not limited to prevention or reduction in interaction between BK and B2R in a cancer cell, which in turn decreases the ability of the cancer cell to migrate into vascular tissue. In a specific embodiment the cancer cell is a glioma cell.

### Methods of Diagnosis

The present disclosure provides methods for determining if a subject is suffering from or at risk for a disease state and condition associated with or characterized by increased activity of one or both of BK activity and B2R activity, such as cancer. The cancer may be, for example, brain cancer, an invasive cancer, or glioma.

Also provided is a method of determining the invasive potential of a cancer cell, said method comprising: measuring a property of the cell, said property selected from the group consisting of B2R activity and B2R expression; and comparing the property to a baseline value; wherein the cell is determined to have increased invasive potential if the property is higher than the baseline value.

Some embodiments of the methods disclosed in this section are in vitro methods; other embodiments are in vive methods.

In one embodiment, the methods for diagnosis involve determining the status of a subject with respect to the activity and/or expression at least one of BK and B2R, or the activity and/or expression of a polypeptide regulated by at least one of BK and B2R. The method may further comprise collecting a sample for testing from the subject.

As used herein, a biological sample which is subjected to testing is a sample derived from a subject and includes, but is not limited to, any biological material, such as a bodily fluid. Examples of bodily fluids include, but are not limited to, whole blood, spinal fluid, serum, saliva, tissue infiltrate, pleural effusions, lung lavage fluid, bronchoalveolar lavage fluid, and the like. The biological fluid may be a cell culture medium or supernatant of cultured cells. For example, the sample can be a blood sample or a serum sample.

The sample may be suspected to harbor cancer cells, for example if a tumor is sampled. In some embodiments of the method the sample is obtained from a brain tumor. In a specific embodiment of the method the sample is obtained from a glioma.

The activity or expression determined in the subject (or sample) may be compared to a baseline value. In some embodiments of the method, the baseline value may be a value reflective of activity or expression in a subject who is not suffering from or at risk of the disease state. In other embodiments of the method, the baseline value is a value reflective of activity or expression in a non-invasive cancer; in further embodiments the value may correspond to a certain type of non-invasive cancer, such as non-invasive brain cancer. In still further embodiments the baseline value may reflect a measure of central tendency of a body of data for either individuals not suffering from or at risk of the disease state; or cancers of a certain type (thus the baseline in such embodiments would reflect an average value). In some embodiments of the method the baseline value is a value obtained from the same subject at an earlier time; in such embodiments the method may be used to monitor changes in the status of the subject over time.

The method may further comprise measuring a second property of the cancer cell selected from the group consisting of the activity of glial fibrillary acidic protein (GFAP) or the expression of GFAP, wherein the cell is determined to have increased invasive potential if the second property is decreased compared to a second baseline. It has been unexpectedly observed that invasive gliomas show normal or reduced levels of GFAP expression compared to non-cancerous cells.

GFAP is one of the major intermediate filament proteins of mature astrocytes. It is used as a marker to distinguish astrocytes from other glial cells during development. Mutations in GFAP cause Alexander disease, a rare disorder of astrocytes in the central nervous system. Alternative splicing results in multiple transcript variants encoding distinct isoforms. Isoform 1 is considered the canonical sequence, which is described further here. The protein is 432 residues in length, and has a mass of 49,880 Da. Numerous variants are known, some of which are cataloged in the Uniprot database under GenBank Accession Number P14136 (SEQ ID NO: 10) (the canonical sequence—incorporated herein by reference to enable the reader to identify GFAP). GFAP has been characterized as having several regions: the head region (positions 1-72), rod (positions 73-377), coil 1A (positions 73-104), linker 1 (positions 105-115), coil 1B (positions 116-214), linker 12 (positions 215-230), coil 2A (positions 231-252), linker 2 (positions 253-256), coil 2B (positions 257-377), and tail (positions 378-432). The protein is believed to be phosphorylated at positions 7, 13, 38, 110, and 383. Several mutations and natural variants have been reported. The GFAP gene is located at position 17q 21 in the human genome. The protein is largely conserved between human (SEQ ID NO: 10), chimpanzee (SEQ ID NO: 11), wolf (SEQ ID NO: 12), cattle (SEQ ID NO: 13), mouse (SEQ ID NO: 14), rat (SEQ ID NO: 15), chicken (SEQ ID NO: 16), and zebrafish (SEQ ID NO: 17).

The difference between the activity or expression in the subject or sample and the baseline value must be a measurable difference. In some embodiments of the method, the difference is at least 1.25-fold, 1.5-fold, 2-fold, 5-fold or higher. In some embodiments of the method the difference is a significant difference, meaning that the difference is greater than the expected range of error of the measurement technique.

Assay techniques that can be used to determine levels of expression or activity in a sample are known. Such assay methods include, but are not limited to, radioimmunoassays, reverse transcriptase PCR(RT-PCR) assays, immunohistochemistry assays, in situ hybridization assays, competitive-binding assays, Western Blot analyses, ELISA assays and proteomic approaches, two-dimensional gel electrophoresis (2D electrophoresis) and non-gel based approaches such as mass spectrometry or protein interaction profiling. Assays also include, but are not limited to, competitive and non-competitive assay systems using techniques such as radioimmunoassays, enzyme immunoassays (EIA), enzyme linked immunosorbent assay (ELISA), sandwich immunoassays, precipitin reactions, gel diffusion reactions, immunodiffusion assays, agglutination assays, complement-fixation assays, immunoradiometric assays, fluorescent immunoassays, protein A immunoassays, and immunoelectrophoresis assays. For examples of immunoassay methods, see U.S. Pat. No. 4,845,026 and U.S. Pat. No. 5,006,459 (incorporated by reference herein only to teach such assays).

In an ELISA assay, an antibody is prepared, if not readily available from a commercial source, specific to an antigen, such as, for example, at least one of BK and B2R or a polypeptide regulated by at least one of BK and B2R. In addition, a reporter antibody generally is prepared. The reporter antibody is attached to a detectable reagent such as a radioactive, fluorescent or enzymatic reagent, for example horseradish peroxidase enzyme or alkaline phosphatase. To carry out one embodiment of the ELISA, an antibody specific to the antigen is incubated on a solid support that binds the antibody. Any free protein binding sites on the dish are then covered by incubating with a non-specific protein. Next, the sample to be analyzed is incubated with the solid support, during which time the antigen binds to the specific antibody. Unbound sample is washed out with buffer. A reporter antibody specifically directed to the antigen and linked to a detectable reagent is introduced resulting in binding of the reporter antibody to any antibody bound to the antigen. Unattached reporter antibody is then washed out. Reagents for detecting the presence of the reporter antibody are then added. The detectable reagent is then determined in order to determine the amount of antigen present. In an alternate embodiment, the antigen is incubated with the solid support, followed by incubation with one or more antibodies, wherein at least one of the antibodies comprises a detectable reagent. Quantitative results may be obtained by reference to a standard curve.

In some embodiments of the assay a genetic sample can be obtained. The genetic sample comprises a nucleic acid, such as RNA and/or DNA. For example, in determining the expression of genes mRNA can be obtained from the biological sample, and the mRNA may be reverse transcribed into cDNA for further analysis. Alternatively, the mRNA itself may be used in determining the expression of genes. A genetic sample may be obtained from the biological sample using any techniques known in the art (Ausubel et al. Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989); Nucleic Acid Hybridization (B. D. Hames & S. J. Higgins eds. 1984) each of the foregoing being incorporated herein by reference only to teach obtaining a genetic sample). The nucleic acid may be purified from whole cells using DNA or RNA purification techniques. The genetic sample may also be amplified using PCR or in vive techniques requiring sub-cloning. The genetic sample can be obtained by isolating mRNA from the cells of the biological sample and reverse transcribing the RNA into DNA in order to create cDNA (Khan et al. Biochem. Biophys. Acta 1423:17 28, 1999).

Once a genetic sample has been obtained, it may be analyzed. The analysis may be performed using any techniques known in the art including, but not limited to, sequencing, PCR, RT-PCR, quantitative PCR, restriction fragment length polymorphism, hybridization techniques, Northern blot, microarray technology, and similar techniques. In determining the expression level of a gene or genes in a genetic sample, the level of expression may be normalized by comparison to the expression of another gene such as a well known, well characterized gene or a housekeeping gene (for example, actin). For example, reverse-transcriptase PCR (RT-PCR) can be used to detect the presence of a specific mRNA population in a complex mixture of thousands of other mRNA species. Hybridization to clones or oligonucleotides arrayed on a solid support (i.e., gridding) can be used to both detect the expression of and measure the level of expression of that gene. In this approach, a cDNA encoding an antigen is fixed to a substrate. The substrate may be of any suitable type including but not limited to glass, nitrocellulose, nylon or plastic. At least a portion of the DNA encoding the antigen is attached to the substrate and then incubated with the analyte, which may be RNA or a complementary DNA (cDNA) copy of the RNA, isolated from the sample of interest. Hybridization between the substrate bound DNA and the analyte can be detected and quantitated by several means including but not limited to radioactive labeling or fluorescence labeling of the analyte or a secondary molecule designed to detect the hybrid. Quantitation of the level of gene expression can be done by comparison of the intensity of the signal from the analyte compared with that determined from known standards. The standards can be obtained by in vitro transcription of the target gene, quantifying the yield, and then using that material to generate a standard curve.

Kits for the above methods of diagnosis and methods of determining the invasive potential of a cell are provided. A general embodiment of the kit for determining the invasive potential of a cell contains a means to measure a property of a cell, the property selected from the activity of B2R, the expression of B2R, the activity of a polypeptide regulated by B2R, and the expression of a polypeptide regulated by B2R. The measurement means may be any described above as suitable to measure expression or activity or other such means known in the art. In more specific embodiments the property is selected from the activity of B2R and the expression of B2R. The measurement means may be, for example, an antibody or antibody fragment that recognizes any of B2R or a polypeptide regulated by B2R. Antibodies and antibody fragments have the advantage of yielding quick results, for example when used in an ELISA assay. A specific embodiment of the kit is an ELISA kit.

### Assays

Methods of determining whether a candidate compound modulates cellular migration are provided. Some embodiments of the method identify compounds that reduce cellular migration; such compounds may be useful as active agents in the pharmaceutical composition described herein. The methods determine the effect of a candidate compound on an assay target. In this context the term “assay target” refers to B2R, BK, a polypeptide regulated by B2R, or a polypeptide regulated by BK. In certain embodiments the assay target is B2R; in other embodiments the assay target is BK. Of course, a given method may determine the effects of a candidate compound on more than one assay target.

A general embodiment of the method comprises: contacting the candidate compound to the assay target; and determining binding between the candidate compound and the assay target; wherein the candidate compound is identified as a modulator of cellular migration if it is determined that the candidate binds to the assay target. Another general embodiment of the method comprises: contacting the candidate compound to the assay target; measuring the activity of the assay target; and comparing the activity of the assay target to a baseline value of the assay target's activity; wherein the candidate compound is identified as a modulator of cellular migration if the activity of the assay target differs from the baseline value. Another general embodiment of the method comprises: contacting the candidate to a cell comprising an assay target selected from B2R and a polypeptide regulated by B2R; measuring at least one property of the assay target selected from the group consisting of expression and activity; and comparing the property of the assay target to a baseline value for the property; wherein the candidate compound is identified as a modulator of cellular migration if the property differs from the baseline value.

The baseline value will reflect the activity or expression of the assay target in the absence of the candidate compound, but otherwise under similar conditions to those of the assay.

In general, such screening methods involve an assay system (as described in more detail below) that expresses at least one assay target, introducing into the assay system a candidate compound to be tested and determining whether the candidate compound binds to the assay target, or modulates the activity of the assay target. Such inhibition or modulation may act directly on the activity the assay target or may be an inhibition or modulation of expression.

Candidate compounds are tested using a variety of assays, such as, but not limited to, assays that employ cells which express the assay target on the cell surface or a polypeptide (cell-based assays); or in assays with isolated assay target (cell-free assays). It should be noted that as BK is a secreted protein, in a certain embodiment of the cell-based assay the assay target is not BK. The various assays can employ a variant of the assay target (e.g., full-length, a biologically active fragment, or a fusion protein which includes all or a portion of the desired polypeptide). Moreover, the assay target can be derived from any suitable species, and may be obtained from a transgenic organism. Such an organism may be a mammal, for example a common animal model such as mice, rats, other rodents, rabbits, dogs, cats, swine, and cattle.

Where the assay involves the use of a whole cell, the cell may either naturally express the assay target or may be genetically modified to express the same. In the latter case, cells can be genetically modified through conventional molecular biology techniques, such as by infecting the cell with a virus comprising a nucleic acid encoding the assay target, wherein the assay target is expressed in the cell following infection. The cell can also be a prokaryotic or eukaryotic cell that has been transfected with a nucleotide sequence encoding the assay target. In the foregoing, full length polypeptides, fragments or fusion proteins containing at least a part of such polypeptide may be used.

The assay can be a binding assay entailing direct or indirect measurement of the binding of a candidate compound to the assay target. The assay can also be an activity assay entailing direct or indirect measurement of the activity of the assay target. The assay can also be an expression assay entailing direct or indirect measurement of the expression of mRNA or protein.

The various screening assays may be combined with an in vive assay entailing measuring the effect of the candidate compound on the symptoms of the disease states and conditions discussed herein. In such an embodiment, the candidates may be evaluated to determine the impact of a parameter associated with the action of the assay target. Such parameters include, but are not limited to, determining the rate of cellular migration or invasion.

### Binding Assays

In one embodiment, the present disclosure provides assays for screening candidate compounds which bind to or modulate the activity of a membrane-bound (cell surface expressed) form of the assay target. Such assays can employ the full-length the assay target, a biologically active fragment of the assay target, or a fusion protein which includes all or a portion of the assay target. The assay target may be expressed in a whole cell or in a liposome, micelle or similar lipid containing structure.

Determining the ability of the candidate compound to bind to a membrane-bound form of the assay target can be accomplished, for example, by coupling the candidate compound with a radioisotope or enzymatic label such that binding of the candidate compound to the assay target-expressing cell can be measured by detecting the labeled candidate in a complex.

For example, the candidate compound can be labeled with ¹²⁵I, ³⁵S, ¹⁴C, or ³H, either directly or indirectly, and the radioisotope detected by direct counting of radio-emission or by scintillation counting. Alternatively, the candidate compound can be enzymatically labeled with, for example, horseradish peroxidase, alkaline phosphatase, or luciferase, and the enzymatic label detected by determination of conversion of an appropriate substrate to product.

In a competitive binding format, the assay comprises contacting the assay target-expressing cell or liposome with a known compound which binds to the assay target to form an assay mixture, contacting the assay mixture with a candidate compound, and determining the ability of the candidate compound to interact with the assay target-expressing cell, wherein determining the ability of the candidate compound to interact with the assay target-expressing cell comprises determining the ability of the candidate compound to preferentially bind the assay target-expressing cell as compared to the known compound.

### Inhibition of Signaling

In another embodiment, the assay is a cell-based assay comprising contacting a cell expressing a membrane-bound form of the assay target (a full-length assay target, a biologically active fragment of the assay target, or a fusion protein which includes all or a portion of the assay target) expressed on the cell surface with a candidate compound and determining the ability of the candidate compound to inhibit the activity of the membrane-bound form of the assay target. Determining the ability of the candidate compound to inhibit the activity of the membrane-bound form of the assay target can be accomplished by any method suitable for measuring the activity of the assay target or the activity of a G-protein coupled receptor or other seven-transmembrane receptor. The activity of a seven-transmembrane receptor can be measured in a number of ways, not all of which are suitable for any given receptor. Among the measures of activity are: alteration in intracellular Ca²⁺ concentration, activation of phospholipase C, alteration in intracellular inositol triphosphate concentration, alteration in intracellular diacylglycerol concentration, and alteration in intracellular adenosine cyclic 3′,5′-monophosphate concentration.

Determining the ability of the candidate compound to modulate the activity of at least one of BK and B2R can be accomplished, for example, by determining the ability of at least one of BK and B2R to bind to or interact with a target molecule, such as a polypeptide regulated by the BK or B2R receptor. The target molecule can be a molecule with which at least one of BK and B2R binds or interacts with in nature, for example, a molecule on the surface of a cell which expresses at least one of BK and B2R, a molecule on the surface of a second cell, a molecule in the extracellular milieu, a molecule associated with the internal surface of a cell membrane or a cytoplasmic molecule. The target molecule can be a component of a signal transduction pathway which facilitates transduction of an extracellular signal (a signal generated by binding of a BK or B2R ligand) through the cell membrane and into the cell. The target molecule can be, for example, a second intracellular protein which has catalytic activity or a protein which facilitates the association of downstream signaling molecules with at least one of BK and B2R.

Determining the ability of at least one of BK and B2R to bind to or interact with a target molecule can be accomplished by one of the methods described above for determining direct binding. In one embodiment, determining the ability of at least one of BK and B2R to bind to or interact with a target molecule can be accomplished by determining the activity of the target molecule. For example, the activity of the target molecule can be determined by detecting induction of a cellular second messenger of the target molecule (intracellular Ca²⁺, diacylglycerol, IP3, etc.), detecting catalytic/enzymatic activity of the target on an appropriate substrate, detecting the induction of a reporter gene (such as a regulatory element that is responsive to a compound operably linked to a nucleic acid encoding a detectable marker, e.g., luciferase), or detecting a cellular response.

### Cell Free Assays

The present disclosure also includes cell-free assays. Such assays involve contacting a form of the assay target (full-length, a biologically active fragment, or a fusion protein comprising all or a portion of a desired polypeptide) with a candidate compound and determining the ability of the candidate compound to bind to the assay target or to inhibit the assay target. Binding of the candidate compound to the assay target can be determined either directly or indirectly as described above. Regulation of the assay target can be determined as discussed above.

In one embodiment, the assay includes contacting a cell free system containing the assay target with a known compound to form an assay mixture, contacting the assay mixture with a candidate compound, and determining the ability of the candidate compound to interact with the assay target, wherein determining the ability of the candidate compound to interact with the assay target comprises determining the ability of the candidate compound to preferentially bind to the assay target as compared to the known compound.

The cell-free assays of the present disclosure are amenable to use of either a membrane-bound form of the assay target or a soluble fragment thereof. In the case of cell-free assays comprising the membrane-bound form of the polypeptide, it may be desirable to utilize a solubilizing agent such that the membrane-bound form of the polypeptide is maintained in solution. Examples of such solubilizing agents include but are not limited to non-ionic detergents such as n-octylglucoside, n-dodecylglucoside, n-dodecylmaltoside, octanoyl-N-methylglucamide, decanoyl-N-methylglucamide, Triton X-100, Triton X-114, Thesit, Isotridecypoly (ethylene glycol ether) n,3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In various embodiments of the above assay methods, it may be desirable to immobilize the assay target to facilitate separation of complexed from uncomplexed forms of one or both of the polypeptides, as well as to accommodate automation of the assay. Binding of a candidate compound to the assay target or interaction of the assay target in the presence and absence of a candidate compound can be accomplished in any vessel suitable for containing the reactants.

Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase (GST) fusion proteins or glutathione-S-transferase fusion proteins can be adsorbed onto glutathione sepharose beads or glutathione derivatized microtitre plates, which are then combined with the candidate compound and the mixture incubated under conditions conducive to complex formation (for example at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of the assay target can be determined using standard techniques.

### Genetic Expression

The screening assay can also involve monitoring the expression of the assay target. For example, regulators of expression of the assay target can be identified in a method in which a cell is contacted with a candidate compound and the expression of the assay target or mRNA encoding the foregoing in the cell is determined. The level of expression of polypeptide or mRNA the presence of the candidate compound is compared to the level of expression in the absence of the candidate compound. The candidate compound can then be identified as a regulator of expression of the assay target based on this comparison. For example, when expression of polypeptide or mRNA protein is decreased in the presence of the candidate compound compared to its absence, the candidate compound is identified as an inhibitor of polypeptide or mRNA expression. The level of polypeptide or mRNA expression in the cells can be determined by methods described below.

The level of mRNA or polypeptide expression in the cells can be determined by methods well known in the art for detecting mRNA or polypeptide. Either qualitative or quantitative methods can be used. The presence of polypeptide assay targets can be determined, for example, using a variety of techniques known in the art, including immunochemical methods such as radio-immunoassay, Western blotting, Northern blots, Southern blots, microarray testing, PCR techniques, including but not limited to, real-time PCR and immunohistochemistry. Alternatively, polypeptide synthesis can be determined in vivo, in a cell culture, or in an in vitro translation system by detecting incorporation of labeled amino acids into the assay target.

Such screening can be carried out either in a cell-free assay system or in an intact cell. Any cell which expresses the assay target can be used in a cell-based assay system. The polynucleotide can be naturally occurring in the cell or can be introduced using techniques such as those described above. Either a primary culture or an established cell line can be used.

### Candidate Compounds

Suitable candidate compounds for use in the screening assays can be obtained from any suitable source, such as conventional compound libraries. The candidate compounds can also be obtained using any of the numerous approaches in combinatorial library methods known in the art, including: biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, non-peptide oligomer or small molecule libraries of compounds. Examples of methods for the synthesis of molecular libraries can be found in the art. Libraries of compounds may be presented in solution or on small particles such as beads, bacteria, spores, plasmids, or bacteriophage.

### Modeling Compounds

Computer modeling and searching technologies permit identification of compounds, or the improvement of already identified compounds, that can inhibit the assay target (either through expression or activity). Having identified such a compound, the active sites or regions are identified. Such active sites might typically be ligand binding sites. The active site can be identified using methods known in the art including, for example, from the amino acid sequences of peptides, from the nucleotide sequences of nucleic acids, or from study of complexes of the relevant compound or composition with its natural ligand.

In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined. If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate compounds can be identified by searching databases containing compounds along with information on their molecular structure. Such a search seeks compounds having structures that match the determined active site structure and that interact with the groups defining the active site. Such a search can be manual, but is may be computer assisted. Alternatively, these methods can be used to identify improved compounds known in the art or identified in one of the screening assays above. These compounds found may be used modulate the assay target.

The present disclosure also provides kits for carrying out any method of the present disclosure, which can contain any of the compounds and/or compositions disclosed herein or otherwise useful for practicing a method of the disclosure.

### Cancer Models

The disclosure provides organisms that are useful as models of cancer, in which the organism has been genetically modified to modulate at least one of the activity or expression of a modulated polypeptide selected from the group consisting of: B2R, BK, a polypeptide regulated by B2R, and a polypeptide regulated by BK. The model may be a model of brain cancer, of an invasive cancer, or of glioma. The organism is not a human being, although it may be a human cell.

In a general embodiment, the organism is a cancer cell, and the modulated polypeptide is selected from the group consisting: B2R and a polypeptide regulated by B2R. In a specific embodiment of the cancer cell the modulated protein is B2R. In a further embodiment the activity or expression of the modulated polypeptide is increased to generate a highly invasive cancer cell. In another embodiment the activity or expression of B2R is decreased to generate a less invasive cancer cell. The cancer cell may be a human cancer cell (for use as a xenograft in an animal model, for example) or an animal cancer cell. Animal cells have the advantage of being less likely to elicit an immune response in an animal model. Human cells have the advantage of more closely emulating human cancer. The cancer cell may be, for example, a glioma cell.

In some embodiments of the organism the expression or activity of the modulated polypeptide has been decreased. Such cells may be valuable in various applications. One exemplary embodiment of the organism in which the modulated polypeptide has been decreased is a genetically modified animal model that is relatively resistant to invasive cancer. The decrease may be achieved by genetic modification of the animal. Examples include deleting a gene for the target molecule, the use of anti-sense RNA, and the use of small interfering RNA. Other approaches may be used as understood in the art.

The modulation in expression or activity may be achieved by any technique of genetic modification known in the art. For example, multiple copies of a structural gene of the modulated polypeptide may be introduced into the cell to increase expression. The structural gene may be a gene that encodes BK or B2R. Embodiments of a structural gene encoding B2R may encode the polypeptide of any one of SEQ ID NO: 2-9, or a conservative variant thereof. The organism would in such a case comprise a nucleic acid comprising at least two copies of the modulated polypeptide operably linked to a promoter. In another example a structural gene for a modulated polypeptide is operably linked to a promoter that is more active than the endogenous promoter. The promoter that is more active than the endogenous promoter may be, for example, a constitutive promoter; in other embodiments it may be an inducible or repressible promoter. In another example, the structure of the polypeptide is varied to increase or decrease activity; in a specific example, the activity is the ability of B2R to bind to BK.

Another general embodiment of the model is a non-human animal comprising an externally administered activator of the modulated polypeptide. Methods are provided to inducing cancer in animal models, comprising administering to the animal an activator of the modulated polypeptide in an effective amount.

### WORKING EXAMPLES

In this study, it is shown that BK, acting via B2R, promotes migration of glioma cells. Low concentrations of BK stimulate sustained increases in intracellular Ca₂ concentration, whereas prolonged exposure to BK induces Ca₂ oscillations in glioma cells which, in turn, significantly enhance cell motility. More importantly, invasion of glioma cells into brain slices and association with blood vessels was disrupted when B2R were pharmacologically inhibited or specific short-hairpin RNA (shRNA) constructs were used. These data suggest that glioma cells use B2R to sense BK cleaved by endothelial cells and use this signal to identify and connect with blood vessels as they invade.

### Material and Methods

Cell Culture

Experiments were done using the glioma cell lines D54-MG [World Health Organization (WHO) Grade IV, glioblastoma multiforme (GBM) provided as a gift by Dr. D. Bigner (Duke University, Durham, N.C.), STTG-1, U251-MG, U87-MG [GBM, WHO grade 4, American Tissue Culture Collection (ATCC)], and two patient-derived acute GBM cultures labeled GBM 50 and GBM 62. The cells were maintained in Dulbecco's Modified Eagle Medium/Ham's F-12 50/50 Mix (DMEM/F12) containing 2 mM glutamine (media and glutamine supplied by Media Tech, University of Alabama at Birmingham Media Preparation Facility) and 7% Fetal Bovine Serum (FBS) (Aleken Biologicals, Texarkana, Ark.), at 37° C. and 10% CO₂. D54-EGFP MG cells were used for transfections with shRNA plasmids (Open Biosystems, Huntsville, Ala.) and clones of the inducible plasmids were generated using puromycin resistance. The human glioma cell line U251-MG [glioblastoma multiforme (GBM), World Health Organization (WHO) grade IV](a gift from Dr. Yancey Gillespie at the University of Alabama at Birmingham) was used to generate U251-MG-GFP cells. Unless otherwise stated, all reagents were purchased from Sigma Aldrich, St. Louis, Mo. In all experiments, cells were treated with one or both B2R antagonists HOE-140 and Bradyzide (BZ).

Western Blotting

For Western blot analysis, non-nuclear membrane enriched protein preparations were obtained from confluent dishes of cells, and processed as previously described (Montana et al., 2004). Protein concentrations were quantified using the DC protein assay kit (Biorad, Hercules, Calif.). 20 μg of protein was aliquoted and 6× Laemmli-SDS sample buffer containing 600 mM β-Mercaptoethanol was added to appropriate proportions and samples loaded into individual lanes of 4-20% pre-cast SDS-PAGE gels (Biorad, Hercules, Calif.). Protein separation was accomplished by 100 V for ˜90 minutes. Gels were then transferred at 350 mA for 90 min at room temperature onto PVDF paper (Millipore, Bedford, Mass.). Membranes were blocked in blocking buffer consisting of 5% nonfat milk in TBS-T. Primary antibodies for B2R (BD Transduction Laboratories, San Jose, Calif.) were diluted in blocking buffer at 1:250 overnight at 4° C. followed by 3 washes. Membranes were incubated with HRP-conjugated secondary antibodies for 1 hour and washed 3 times. The blots with HRP-conjugated secondary antibodies were developed using FemtoWest (Pierce, Rockford, Ill.) and all blots were imaged on a Kodak 4000MM imager (Rochester, N.Y.).

Immunocyto/Histochemistry

D54-MG, STTG1, U251-MG, U87-MG, GBM 62 and GBM 50 cells were seeded on glass coverslips (12 mm round, Macalster Bicknell, New Haven, Conn.) and, once they reached 70% confluency, fixed in 4% paraformaldehyde for 15 minutes and rinsed at room temperature. Cells were permeabilized with 0.25% Triton X-100 in phosphate buffered saline (PBS), and then blocked in PBS with 0.25% Triton X-100 and 5% goat serum for 30 minutes at room temperature. Primary B1R and B2R rabbit polyclonal antibodies were obtained from Sigma, 1:100 dilution in PBS with 5% goat serum, were incubated at 4° C. overnight. The following day, cells were rinsed three times with PBS. TRITC-conjugated goat anti-rabbit secondary, 1:750, (Molecular Probes, Eugene, Oreg.) in PBS and 5% goat serum were incubated on cells in the dark for 1 hour at room temperature. Cells were then washed once with PBS, incubated 5 min with DAPI diluted 1:2000 in PBS, and washed twice more with PBS. Coverslips were then mounted on glass slides with Gel Mount Aqueous Mounting Medium, and imaged. For immunohistochemistry, 100 μm sections of human tissue biopsies were fixed in 4% paraformaldehyde for 2 hours at room temperature. Following triple washes with PBS, samples were permeabilized for 1-2 hours with PBS with 0.25% Triton X-100, blocked in PBS with 0.25% Triton X-100 and 5% goat serum for 2 hours at room temperature, and incubated in primary antibodies overnight at 4° C. In subset of experiments, tissue sections were double-labeled. Antibodies against laminin (1:500) and glial fibrillary acidic protein (GFAP) -Cy3 (1:400), both obtained from Sigma, were used. FITC- and TRITC-conjugated goat anti-rabbit secondary, 1:750, (Molecular Probes, Eugene, Oreg.) in PBS and 5% goat serum were incubated on cells in the dark for 2 hour at room temperature. Following staining, the slices were mounted in between two coverslips for imaging. Quantification of fluorescent staining intensity after background subtraction was analyzed using Slidebook 4.2 software (Intelligent Imaging Innovations, Denver, Colo.). All experiments were repeated at least three times.

Calcium Imaging

D54-MG cells were plated on 35-mm glass bottom dishes (MatTek, Corp., Ashland, Mass.) at ˜100×10³ per dish and cultured for 2 days. pTRIPZ transfected cells, after stable selection, were plated at a density of ˜20×10³ per dish and treated with 1 mg/mL doxycycline for 4 to 5 days to allow for sufficient protein knockdown. All cells were loaded in serum-free culture medium for 45 min with the ratiometric Ca²⁺ dye Fura-2-acetoxymethylester (5 μmol/L; TEFLABS, Austin, Tex.) reconstituted in 20% wiv pluronic acid in DMSO (Invitrogen, Carlsbad, Calif.). Cells were rinsed with serum-free medium and allowed to rest in 7% serum-containing medium for 45 min at 37° C. The glass-bottom dishes were placed in an environmental chamber mounted on an inverted microscope. Cells were allowed to equilibrate in the chamber for 15 min before calcium images were collected. Following equilibration, recordings were obtained with an Olympus Disk Spinning Unit (DSU) fluorescent imaging microscope where cells were alternately excited at 340 and 380 nm using an x-cite illumination light source. Emitted light was collected at >520 nm. Images were digitized online using Slidebook 4.2 software (Intelligent Imaging Innovations, Denver, Colo.), and 340:380 nm ratios were obtained every 15 seconds. Following a 5 minute baseline recording every 15 seconds, BK was applied to the cells and imaging continued every 15 seconds for 30 or 60 minutes. In a subset of experiments, BK antagonists, HOE-140 and Bradyzide were applied to the cells. These experiments were repeated at least three times and data were pooled for statistical analysis.

Time-Lapse Motility Assay

Time-lapse studies were performed using a stably transfected daughter cell line from D54-MG cells that express EGFP, previously generated in the lab (Habela et al., 2009) and U251-EGFP cell line to visualize cells as they migrate. Increased motility was tested using an Ibidi chemotaxis chamber that is commercially available from Ibidi Scientific (Ibidi GmbH, Martinsried, Germany), and which has been used in previous studies (Kronlage et al., 2010; Fabian et al., 2010) with D54-EGFP MG cells seeded. Liquid chemical gradients were established in the μ-channel of the μ-chamber as follows: the dye Patent Blue V Blue Sodium Salt (1 mg/ml in medium) was mixed with equivolume of medium+/−1 mM stock of BK+/−1 mM stocks of HOE-140 or BZ; half of the volume was loaded in the well at one side of the μ-channel, and the identical volume was then aspirated from the well on the other side of the μ-channel. This created concentration gradient easily visualized by the dye. 12-15 different positions along the concentration gradient were imaged every 5 minutes over 5 hours period on a Zeiss Axiovert 200M system equipped with a Zeiss micro-incubation chamber that maintains cells at constant temperature (37° C.) and 95%/5% CO₂/O₂ for extended periods of time. We obtained images from different sections using 20× objective and FITC filter, along with DIC images. 500× neutral density filters and capture images using Axiovision software and a digital Axiocam HR 1 megapixel camera in 2*2 binning mode was used. Data was analyzed using the imaging tools provided by National Institutes of Health Image J and Tracking, and Chemotaxis and Migration Tool plugins (Jacobelli et al., 2009; Coller et al., 2009; Kronlage et al., 2010; Fabian et al., 2010) by randomly selecting 15 cells for each field of view. Cells treated with BK+/−antagonists were compared with controls where only dye was added. These experiments were repeated in triplicates.

Migration/Invasion Assay

The day before the experiment was performed, ˜70% confluent dishes of cells were prepared. Transwell migration Fluoroblock cell culture inserts (BD Biosciences, San Jose, Calif.) with 8 μm pores were coated overnight with Vitronectin (BD Biosciences, San Jose, Calif.) at a concentration of 5 μg/ml in PBS. The following day, inserts were blocked with 1% fatty acid-free bovine serum albumin for 1 h. Inserts were then washed two times in PBS, and 400 μl of Migration Assay Buffer (MAB, 0.1% fatty acid-free bovine serum albumin in serum-free media) was added to the bottom of each well. Cells were rinsed once in PBS and were lifted off the dish by the addition of 0.5 mm EGTA. Cells were rinsed twice by centrifugation and resuspended in MAB and counted. Forty thousand cells were plated on the top of each filter and allowed to adhere for 30 min before drug was added. When BK+/−antagonists was added to the filters, it was added only to the bottom of the filter for chemotaxis. For migration assay, D54-EGFP MG cells were allowed to migrate for 5 hours, washed with PBS, fixed with 4% paraformaldehyde 15 minutes at room temperature, washed thrice and counterstained with DAPI for 5 minutes at room temperature. Invasion assay was performed similarly. Matrigel cell cultured inserts (BD Biosciences, San Jose, Calif.) with 8 μm pores were pretreated Vitronectin and blocked as described above. 40,000 of D54 cells were plated on the top of each filter, BK+/−antagonist were added to the bottom of the chamber and cells were allowed to invade through artificial, more complex Matrigel matrix barrier for 24 hours. Inserts in which drugs were omitted were processed in parallel and used as controls. Filters were then fixed and stained with Crystal Violet overnight at 4° C., washed with PBS, the tops were wiped clean of cells, and representative fields (five per filter) were imaged with a Zeiss Axiovert 200 M microscope with a 20× objective. The number of nuclei that migrated through pores was counted. All counts per treatment were averaged and SE values were calculated. These experiments were repeated at least thrice and data were pooled for statistical analysis.

In-Situ Zymographic Analysis of Cell Surface Gelatinolytic Activity In Vitro

To assess gelatinolytic MMP activity we used in situ zymography, as described previously (Deshane J et al, 2003). Briefly, after cell nuclei were labeled with Hoechst dye, fluorescein isothiocyanate-labeled DQ gelatin with BK+/−antagonists (Molecular Probes, Eugene, Oreg.) was applied overnight on the coverslips plated with D54 MG cells. Coverslips where drugs were omitted were processed in parallel and used as controls. At the end of the incubation period, cells were photographed by fluorescence microscopy with a Zeiss Axiovert 200 M microscope using a 20× objective. Quantification of fluorescent staining intensity after background subtraction was done using Slidebook 4.2 software (Intelligent Imaging Innovations, Denver, Colo.). Experiments were repeated three times.

Slice Invasion

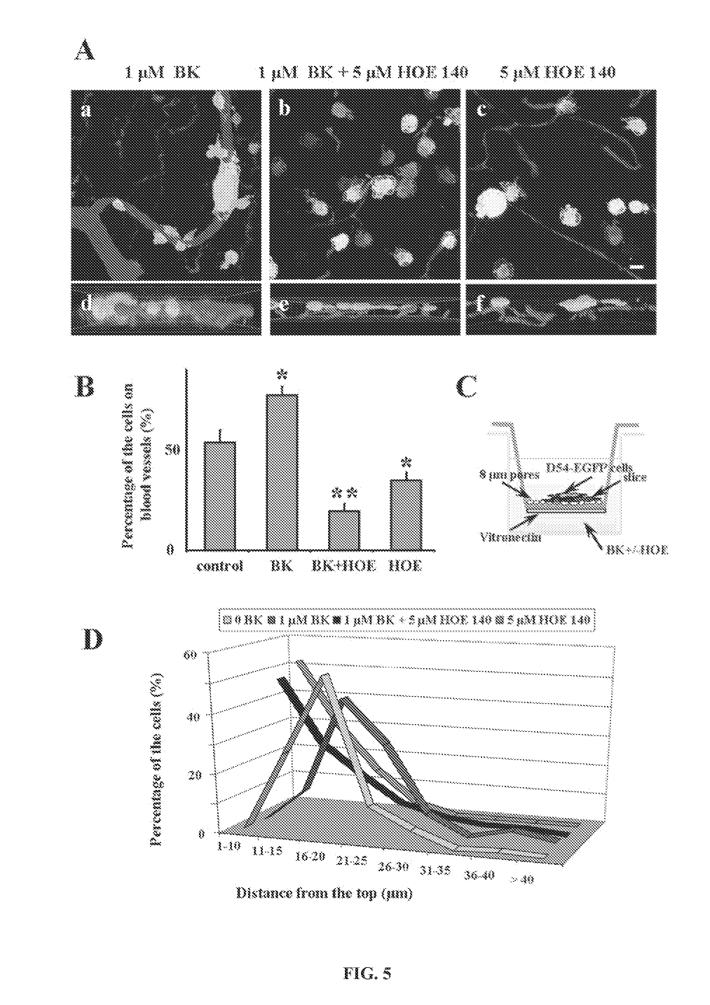

Experiments were performed on male and female Sprague-Dawley rats and were approved by the University of Alabama Institutional Animal Care and Use Committee. 17-22 days old pups were decapitated. Meninges were stripped, brain was taken out and put in ice-cold bath ACSF. Tissue was sliced using Vibrotome 3000 sectioning system. 300 μm thick slices were let recover in ACSF for 1 hour at room temperature, followed by recovery in ACSF at 37° C. in 95%/5% CO₂/O₂ for 1 hour. CD31 antibody (BD Biosciences, Pharminogen, San Diego, Calif.) that labels blood vessels was added during the latter recovery period. Slices were then transferred into transwell migration cell culture inserts (BD Biosciences, San Jose, Calif.) with 8 μm pores that were pretreated with Vitronectin and blocked as described above. 50,000 D54-EGFP MG cells were then seeded on top of the slices and allowed to migrate/invade into the tissue for 2 hours at 37° C. During that period, slices were treated with 1 μM BK, 1 μM BK+5 μM HOE-140 or 5 μM HOE-140 added to the bottom of migration chamber in order to create a concentration gradient, similar to in vitro migration/invasion assay (FIG. 5C). In control experiments, drug treatment was omitted. Following double washes with PBS, slices were fixed in 4% paraformaldehyde overnight at 4° C. The following day, samples were washed three times with PBS and the slices were mounted between two coverslips for imaging on an Olympus Fluoview confocal microscope with 60× objective.

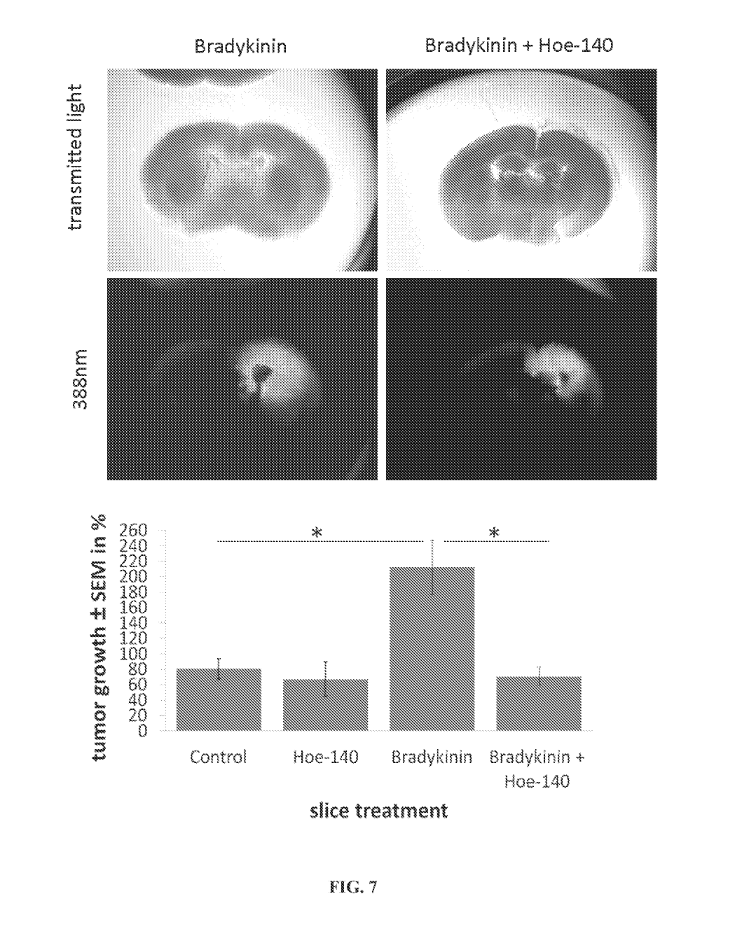

Slice cultures were prepared from brains of P13-P16 BALB/c scid mice (Jackson Laboratory). Coronal brain sections 300 μm thick were sliced with a Vibrotome 3000 sectioning system in ice cold artificial saline. Brain slices were transferred into filter inserts with a polycarbonate membrane (Falcon, BD, pore size 0.45 μm). Filters were placed into 6-well plates containing 1 ml of DMEM supplemented with 8% FCS, 0.2 mM glutamine, 100 U/mL penicillin, and 100 mg/mL streptomycin. After overnight repose the medium was changed to cultivation medium containing 25% heat-inactivated horse serum, 50 mM sodium bicarbonate, 2% glutamine, 25% Hank's balanced salt solution, 1 mg/mL insulin (all from Gibco), 2.46 mg/mL glucose (Sigma Aldrich), 0.8 mg/mL vitamin C (Sigma Aldrich), 100 U/mL penicillin, 100 mg/mL streptomycin (Sigma Aldrich), and 5 mM Tris in DMEM (Gibco). At day 3 of culturing approximately 3000 D54-EGFP tumor cells in a volume of 1 μl were implanted per organotypic brain slice. Cells were injected using a 1 μl hemilton syringe mounted to a micromanipulator. The cell suspension was slowly injected over 30 seconds and subsequently the syringe was slowly pulled out. Gliomas were always inoculated into the right cortex. Left side of the brain slice was used for control purposes. Treatment of brain slices was started at day 3 of culturing. Slice culture medium was supplement with nothing (n=14), bradykinin (1 μM) (n=13), HOE-140 (5 μM) (n=4) or bradykinin and HOE-140 (n=9) (all from Tocris Bioscience). The slice medium was exchanged every two days containing the drugs. Tumors were photographed at day 4 and day 11 with a Leica MZ 120 Microscope (FIG. 7). The tumor area was determined using ImageJ software (available from rsbweb.nih.gov). Tumor growth was calculated between day 4 and day 11 of culturing.

Transfections of shRNA and Control Plasmids

For inducible B2R knockdown in D54-EGFP MG cells, pTRIPZ-lentiviral vectors were obtained (Open Biosystems, Huntsville, Ala.; catalog numbers RHS4743 and RHS4696-99682, RHS4696-99635991, RHS4696-99408793) for NS, shRNA1 and shRNA2 plasmids respectively, and TurboRed® expression indicated induction of shRNA. Cells were transfected as described in (Weaver et al., 2006). To generate stable lines, 1 ug/mL puromycin treatment began 96 hours after transfection. After selection, cells were passed (density: 0.5 cells/100 μL) into 96 well plates and scored for single colonies. Cells were treated with doxycycline and B2R knockdown was assessed.

Data Analysis

Results were analyzed using Origin (v.6.0, MicroCal Software, Northhampton, Mass.). Significance was determined by one-way ANOVA or Student t-test, as appropriate, since all data showed normal distribution. Post-hoc comparisons were performed using Tukey analysis. All data reported are mean±S.E.M. and * denotes significance p<0.05, ** p<0.01 and *** p<0.001.

### Results

Expression of B2R in Glioma Cell Lines and Patient Tissue Biopsies.

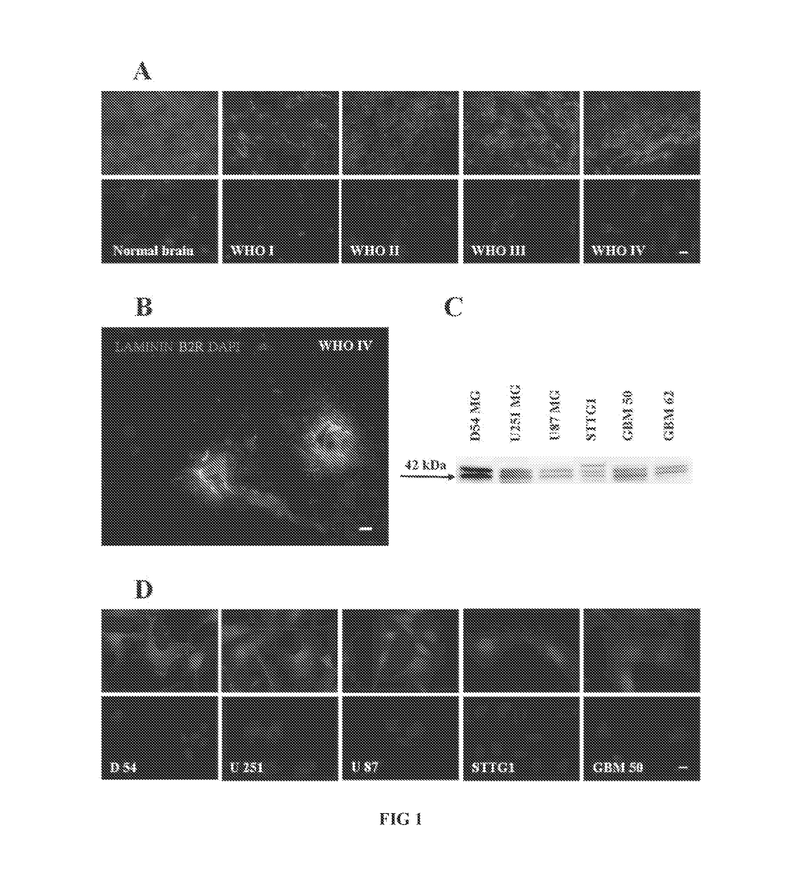

While BK can bind to two classes of receptors, B1R and B2R, previous studies reported increased expression of B2R in glioma biopsy tissues (Raidoo et al., 1999). FIG. 1A shows representative examples of patient derived tissue sections stained for B2R showing immunoreactivity across all four malignancy grades (WHO grades I-IV) as well as in normal brain. Indeed, normal brain samples typically showed uniform B2R immunoreactivity (FIG. 1A) and co-labeling with GFAP antibodies (Supplemental FIG. 1) indicate significant co-localization, suggesting that B2R is expressed in normal astrocytes. Increasing grades of malignancy showed increasing B2R immunoreactivity yet also presented a decrease in GFAP expression. Indeed, in Grade IV samples we observed areas with strong B2R expression that lacked GFAP immunoreactivity (Supplemental FIG. 1). Importantly, we found highest levels of BR2 immunoreactivity in perivascular regions as shown in a representative example in FIG. 1B. Here we co-labeled the tissue with anti-laminin antibody to label blood vessels.

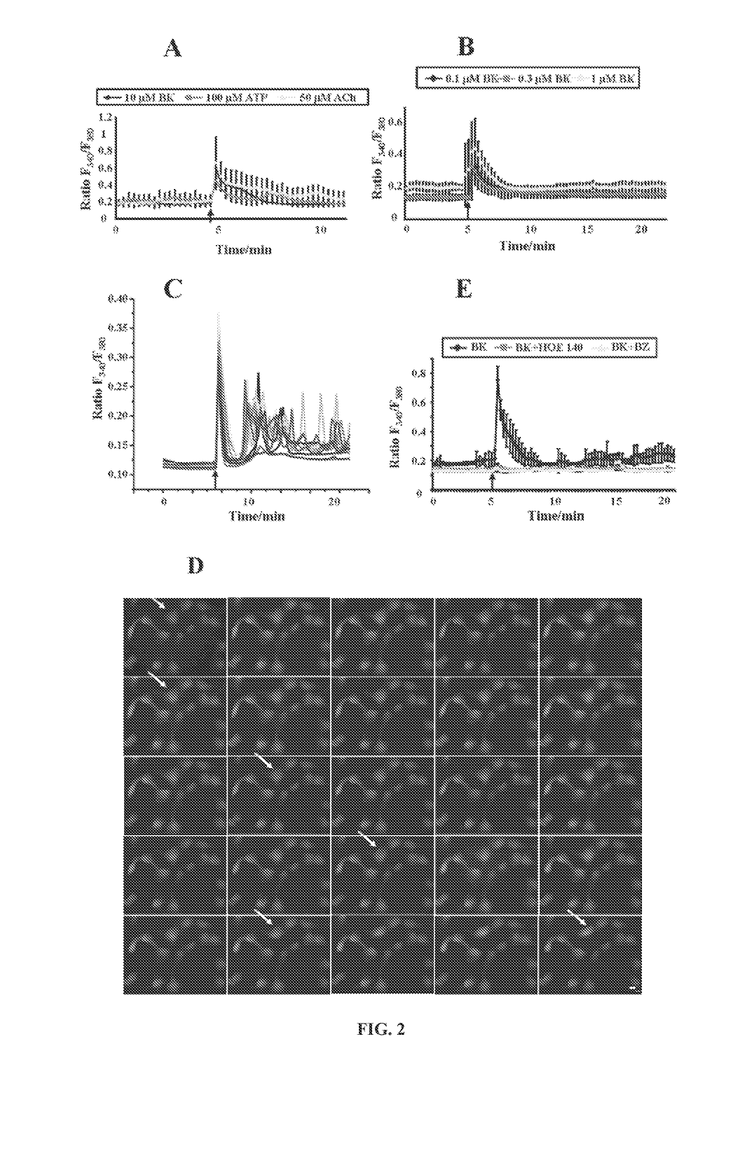

Expression of B2R is also maintained in many frequently used human glioma cell lines: D54-MG, U251 MG, U87 MG, STTG1 and GBM 50 (FIG. 1D). Representative images of immunostained cells indicate strong membrane-associated labeling with specific antibodies against B2R. Nuclei were visualized with DAPI and we controlled for the background by staining coverslips processed in parallel identically, but without primary antibody (lower panels in 1A and D). B2R protein expression was also confirmed by Western blot analysis in membrane enriched (non-nuclei) protein preparations revealing a characteristic double band where the lower band (42 kDa) corresponds to B2R, as specified by the manufacturer (Ewert et al., 2003) (FIG. 1C). B1R was barely detectable by immunohistochemistry in a side-by-side comparison staining D54-MG with specific antibodies against B1R and B2R and quantitative assessment of fluorescent intensities (Supplemental FIG. 2) suggesting that in our experimental system B2R is the dominant BK receptor expressed.

Glioma Cells Respond to Various Stimuli by Increasing Intracellular Calcium Concentrations, Yet Only Prolonged BK Exposure Resulted in Calcium Oscillations Through Binding to B2R.

Glioma cells respond to environmental clues as they invade the brain. It has been previously shown that the exposure of glioma cells to certain neuroligands induces increase in intracellular calcium concentrations. In this series of experiments, we investigated Ca²⁺-responses in cultured D54-MG glioma cells, after addition of acetylcholine (ACh), ATP or BK. Glioma cells were loaded with the ratiometric Ca²⁺-dye FURA2-AM, and imaged over 30 min. Application of either 50 μM ACh (116 cells responded of 128 cells imaged; average peak 0.23±0.04), 100 μM ATP (87/146 cells responded; average peak 0.45±0.05), or 10 μM BK (85/109 cells responded; average peak 0.45±0.03) each caused a sustained rise in intracellular calcium (FIG. 2A). Similar Ca²⁺-responses were observed with much lower BK concentrations (FIG. 213), namely 0.1 μM (256/283 cells responded; average peak 0.34±0.02), 0.3 μM (300/330 cells responded; average peak 0.33±0.07), and 1.0 μM (231/247 cells responded; average peak 0.30±0.07). Prolonged exposure (60 min) of D54-MG cells to 1 μM BK resulted in Ca²⁺-oscillations (FIG. 2C). Similar Ca²⁺-oscillations have been reported in migratory cerebellar granule cells and correlated with the ability of the cells to migrate (Lyons et al., 2007, Komuro and Rakic, 1996). To illustrate an association of cell movement with Ca²⁺ oscillations we show a sequence of time-lapse images from D54 cells loaded with Fura-2 (FIG. 2D) spanning a 5 minute time period (the corresponding movie is in supplemental data). The cell labeled with the arrow shows large calcium oscillations preceding translocation. To determine if these effects were due to actions of BK (236/269 cells responded; average peak 0.75±0.05) on B2R, these experiments were repeated in presence/absence of the B2R antagonists HOE-140 (22/162 cells responded; average peak 0.15±0.01 which was significantly different from response to BK, p<0.001, One-way ANOVA), and Bradyzide (156/300 cells responded; average peak 0.15±0.02 which was significantly different from response to BK, p<0.001, One-way ANOVA). No increase in intracellular Ca²⁺-concentrations was observed in the presence of these specific B2R antagonists (FIG. 2E),

Glioma Cell Motility Increases in a BK Concentration Gradient.

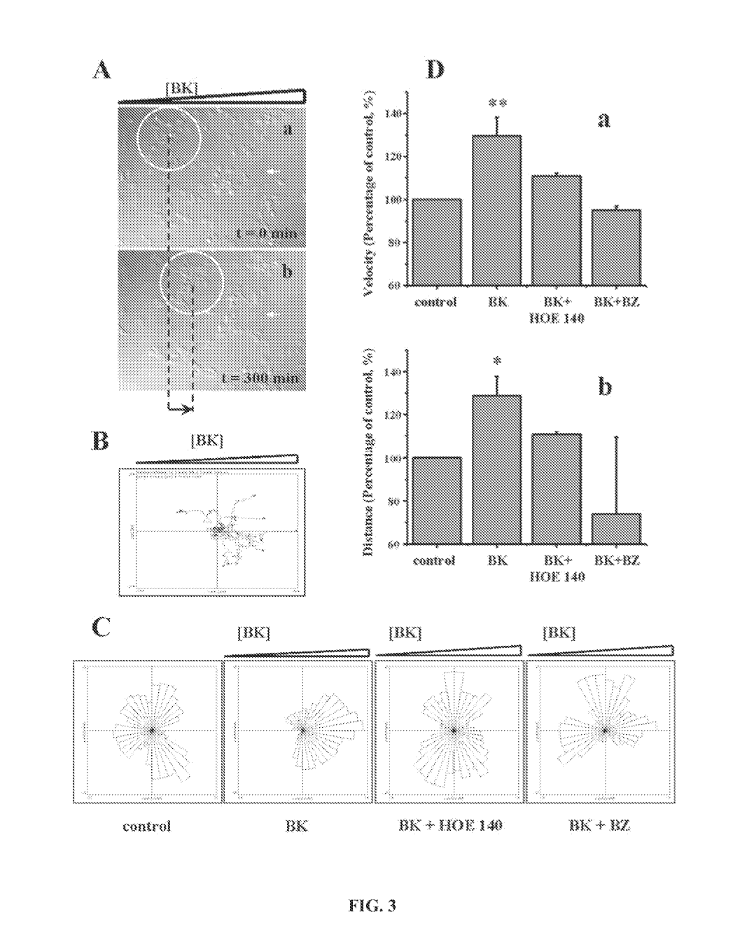

To more closely investigate the responsiveness of glioma cells to BK, the underlying signaling was examined in more detail. As calcium dynamics are often correlated with cell motility time-lapse studies were performed using a stably transfected daughter cell line from D54-MG cells that expresses GFP to visualize cells as they migrate (FIG. 3A). Increased motility was tested in μ-slides in which a BK concentration gradient can be maintained. Multiple fields of view of the cells were imaged during a 5 h period and different migration parameters were analyzed using NIH Image J. A representative example of directionality analysis shown in FIG. 3B, red traces, reveals that in the presence of BK, cells moved towards increasing BK concentration. By contrast, in the absence of a BK concentration gradient, or in the presence of the B2R antagonists HOE-140, and Bradyzide, in addition to BK, cells failed to show any directionality of movement. Averaged cell paths of these examples are featured by rose diagrams in FIG. 3C. Both cell velocity and distance traveled were significantly increased for cells maintained in the BK concentration gradient (29.5±8.7%, 28.8±8.9% respectively) compared to control, while in the presence of specific antagonists, the effect was not observed (FIG. 3C,b and 3C,c respectively; One-way ANOVA, **p<0.01). Another frequently used human glioma cell line was tested, U251-MG that stably express EGFP and obtained similar results (data not shown). Taken together, these data suggest that BK, acting through B2R, increases cell motility and chemo-taxis (a directed movement of a cell or groups of cells towards increased concentrations of the chemical in their environment) in glioma cells.

Transwell Glioma Cell Migration/Invasion Assays Suggest that BK Enhances Invasive Migration of Glioma Cells.

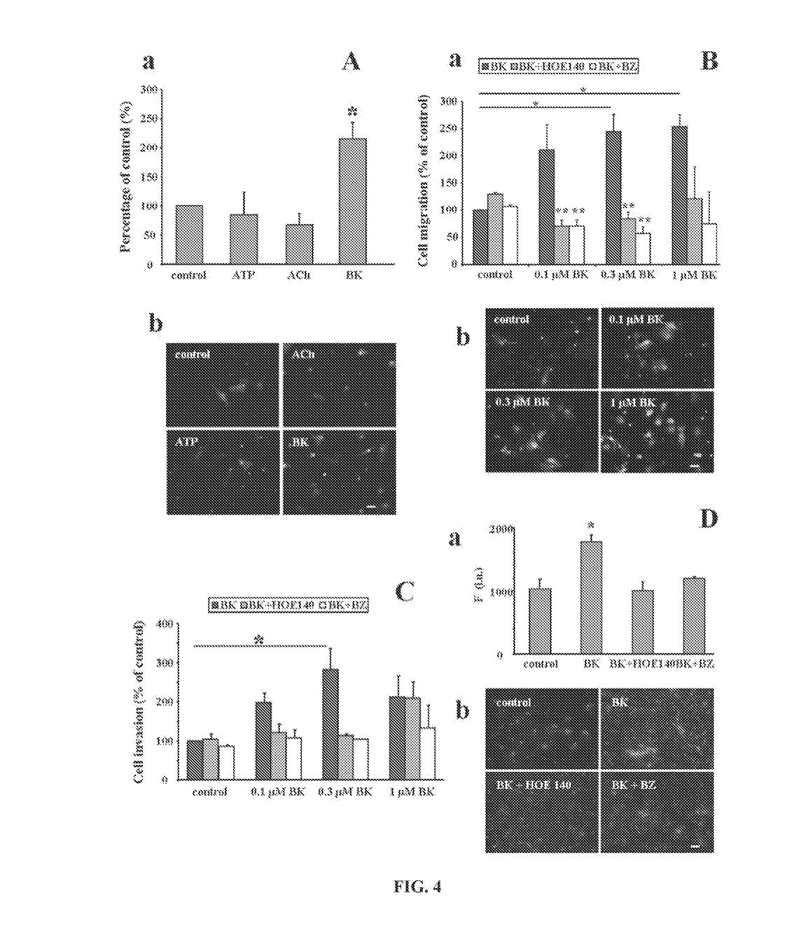

To investigate the hypothesis that BK promotes glioma migration in vitro, we used Transwell migration assay which conveniently permit examination of cell migration across a membrane barrier with 8 μm pores towards vitronectin, a chemo-attractant extracellular matrix protein. This frequently used assay allows a quantitative study of drug effects with regards to drugs acting as motogens or as chemo-attractants. D54-MG-GFP glioma cells were allowed to migrate for 5 h in the presence/absence of neuroligands. First, we tested the effects of ACh, ATP and BK on D54-MG cell migration and found that only BK, which also caused oscillatory changes in Ca²⁺, was able to significantly increase migration (One-way ANOVA, *p<0.05), while ACh and ATP, which did not increase frequency of Ca²⁺ oscillations, failed to alter glioma migration (FIG. 4A, a and A, b). Next, we tested glioma cell migration in various BK concentrations. Parallel experiments using pharmacological antagonists of B2R, HOE-140 and Bradyzide (1 μM), were performed. The data demonstrated a significant increase in the percentage of cells migrating through the pores toward BK (FIG. 4B, a and B, b). This increase was eliminated if either of the B2R antagonists were used. The reduction in migration was significant when compared to parallel experiment where only BK was added (One-way ANOVA, * p<0.05, ** p<0.01). Similar results were obtained when an invasion assay was performed (FIG. 4C). In this assay, cells are exposed to a more complex, artificial Matrigel matrix barrier. We hypothesized that the observed increase was due to activation of MMPs as release of gelatinases MMP2 and MMP9 from glioma cells has been reported (Friedberg et al., 1998). To further investigate the observed increase in invasion activity of D54-MG-GFP in the presence of BK, the ability of BK to induce MMP activity in the presence or absence of the B2R antagonists HOE-140 and Bradyzide was examined. Therefore a MMP gelatinase activity assay was used analyzing changes in enzymatic activity of MMP by BK. A significant increase of fluorescence in the presence of BK indicates increased MMP activity, while addition of the B2R antagonists, HOE-140 and BZ, resulted in reduction of the fluorescence back to control levels (FIG. 4Da and b) (*p<0.05, One-way ANOVA). Taken together, these data suggest that BK, acting through B2R signaling pathway, enhances D54 MG invasion possibly through an increase in MMP activity.

BK Enhances Cell Invasion in Brain Slices.

Having established a dependence of migration and BK in vitro, the role of BK signaling in brain slices was examined, where the presence of neurons, glia and endothelial cells resemble the complex environment more reminiscent of the actual invasions of glioma cells in the human brain. To this end, acute brain slices of rat cortex, that can be maintained viable for many hours of investigation, were incubated with CD31 antibody to label blood vessels and seeded with D54-MG-GFP cells. Cells were allowed to migrate and invade for 2 hours in presence or absence of BK and in presence or absence of B2R antagonist HOE-140. The experimental set-up is illustrated in FIG. 5C. Representative confocal images of glioma cells invading into the slice in the presence of BK with and without antagonist HOE-140 added are shown in FIG. 5A, a, A, b and A, c. The bottom panels in FIG. 5 show cross-sections of reconstructed z-stacks indicating deeper penetration of glioma cells enwrapping blood vessel, 50 μm section in BK bath (FIG. 5A, d), while addition of B2R antagonist retains most of the cells on the top of the slice, 27 μm section, as seen in FIG. 5A, e. Similarly, most of the cells remained on the slices when treated only with B2R antagonist, as depicted in 40 μm cross-section in FIG. 5A, f. Data were analyzed by counting the number of glioma cells that attached to blood vessels in the presence of BK (77.4±4.1%, 391 cells analyzed), BK and antagonist HOE-140 (19.3±3.4%, 461 cells analyzed) or antagonist HOE-140 (42.7±4.2%, 452 cells analyzed) and compared with control (53.5±6.3%, 403 cells analyzed) where no drugs were added. The presence of BK significantly increased the number of glioma cells attached to blood vessels while high doses of antagonist (5 μM HOE-140) significantly reduced the number of glioma cells attached to blood vessels compared to control. Interestingly, when antagonist alone was added, there was also a reduction in the percentage of cells associated with blood vessels suggesting that the antagonist also blocked the effects of endogenous BK present in the slice (FIG. 5B), implicating more complex interactions of glioma cells with their environment. Another important effect observed was that the glioma cells were invading deeper into the tissue in the presence of BK when compared to controls. In the control experiment 27.5% of cells invaded up to 15 μm into the tissue, 53.6 up to 20 μm, 11.9% up to 25 μm; when BK was added 11.9% of the cells invaded up to 15 μm, 44.2% up to 20 μm, 29.6% up to 25 μm; when antagonist HOE-140 was present in addition to BK most of the cells analyzed (47.5%) remained on the top of the slice, 25.7% migrated up to 15 μm, 15.4% up to 20 μm, and 6.4% up to 25 μm; likewise, addition of the antagonist HOE-140 alone left 51.9% cells on top of the slice, 29.8% migrated up to 15 μm, 14.4% up to 20 μm, 3.5% up to 25 μm (FIG. 5D).

In mouse brain slices (FIG. 7), macroscopic imaging of the spread of GFP-expressing tumor cells show significantly reduced migration of tumor cells in the presence of HOE-140 and BK compared to the presence of BK alone (top frame, “388 nm”). Analysis with ImageJ software showed significantly greater tumor growth in slices exposed to BK alone than in slices exposed to BK and HOE-140 (lower frame). Tumor growth in slices exposed to BK and HOE-140 did not differ significantly from growth in control slices or slices exposed to HOE-140 alone.

BK Effects on Glioma Cells are Mediated by B2R.

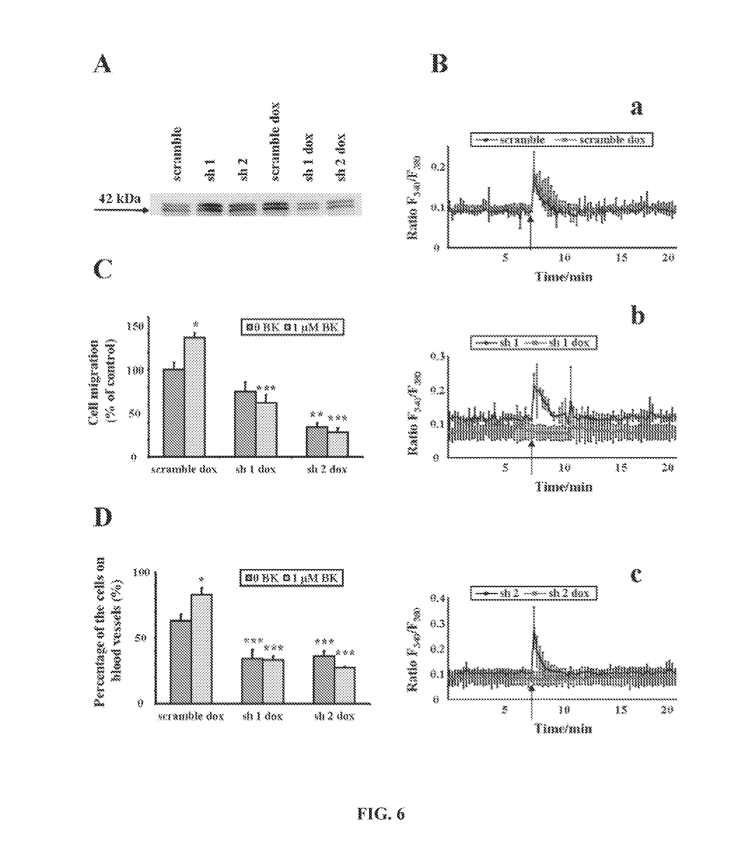

These studies so far pharmacologically manipulated the receptor using two different antagonists. HOE-140 was previously shown to be competitive antagonist very specific to signaling through B2R. However, to additionally confirm that the observed effects of BK on glioma cells were indeed mediated by B2R, D54-MG cells were generated that stably expressed shRNA to suppress the expression of B2R under a doxycycline inducible promoter. A significant reduction of B2R expression in doxycycline induced cells was confirmed by Western blot analysis of non-nuclear membrane protein preparations (FIG. 6A). Next, battery of experiments was performed to test for a functional capability of the remaining expressed protein. Ca²⁺-response in the transfected D54-MG-GFP cells was compared on/off doxycycline. Cells transfected with non-silencing RNA did not show any difference in Ca²⁺-concentrations after addition of BK (96/10⁴ cells responded with average peak 0.17±0.01 which was not significantly different from calcium responses of cells treated with doxocycline, 78/96 with average peak 0.19±0.03, One-way ANOVA). Both cell lines transfected with different silencing shRNAs, lacked any response to BK in doxycycline induced conditions (sh1: 60/73 cells responded with average peak 0.21±0.02 which was significantly different from calcium responses of cells treated with doxycycline, 40/159 with average peak 0.1±0.03, *p<0.05, One-way ANOVA; sh2: 106/125 cells responded with average peak 0.28±0.04 which was significantly different from calcium responses of cells treated with doxycycline, 18/101 with average peak 0.09±0.01, **p<0.01, One-way ANOVA) (FIG. 6B). The migratory abilities of B2R knockdown D54-MG-GFP cells on doxycycline were significantly reduced and were unaltered in the presence of BK (FIG. 6C). Slice invasion assays yielded similar results: the percentage of B2R knockdown D54-MG-GFP cells on doxycycline that attached to the blood vessels was significantly reduced compared to the control and this did not change if BK was added to the system (FIG. 6D). This is in good agreement with the reduction observed due to B2R antagonist effect in slice invasion experiment. Taken together, these data show an important role for B2R in BK mediated chemo-attraction of glioma cells to blood vessels.

### Discussion

Without wishing to be limited to any hypothetic model, certain conclusions can be drawn from the results presented.

This study tested the hypothesis that BK contributes to the invasive migration and dispersal of astrocyte-derived tumors through activation of B2R. B2R expression correlates positively with tumor grade in patient tissue biopsies. Glioma cell lines maintain membrane expression of B2R, as demonstrated by immunocytochemistry and Western blot analysis. The functional assays demonstrate that BK stimulates the migration of glioma cells in vitro and in acute slices, where BK mediates association of invading glioma cells with blood vessels. Perivascular migration is one of three pathways used by glioma cells to disperse and this mode of cell invasion is well recapitulated in the xenograft invasion model used in the studies. Using pharmacological manipulation and inducible B2R knockdown cells we demonstrate that these actions of BK on glioma cells were indeed due to activation of B2R and enhance successful attraction of gliomas to blood vessels.

The B2R is a constitutively active protein localized mostly, as the data demonstrate, on the plasma membrane, although nuclear membrane localization in embryonic rat neurospheres has been reported (Martins et al., 2008). The finding that increased expression of B2R correlates positively with pathological tumor grade in human gliomas is consistent with a previous study (Zhao et al., 2005). The over-expression of the receptor on the membrane implicates a functional importance of this receptor. Indeed, over-expression of B2R on glioma cells has been shown to enhance BK-mediated tumor blood-brain barrier permeability increase (Uchida et al., 2002). A number of later studies further supported this finding by using small concentrations of BK to insure better and more efficient drug delivery to malignant brain tumors (Xia et al., 2009; Zhang et al., 2009; Wang and Liu, 2009; Sarin et al., 2009; Cote et al., 2010). Of note, B2R was also expressed in normal brain, where it associated with GFAP positive astrocytes, yet highest levels of expression were found in gliomas associated with blood vessels. While BK may enhance the delivery of chemotherapeutics to the brain, the data suggest a secondary, undesired effect of BK in enhancing invasion and possibly increasing rather than diminishing the formation of satellite tumors. Of interest, under the experimental conditions, a significant increase was demonstrated in both velocity and distance traveled by the cells when exposed to a BK concentration gradient. Evidently, BK enhances motility of glioma cells. The finding that in vitro migration is significantly increased in the presence of BK compared to control, is in agreement with a recent study in human chondrosarcoma (Yang et al., 2010), suggesting that this effect may apply to other cancers as well. A recent report suggests that BK enhances migration of C6 rat glioma cells and U251 human glioma cells in vitro through B1R activation of PI-3 kinase/AKT signaling cascade (Lu et al., 2010). Our data show only very low expression of B1R, but very prominent expression of B2R, and together with data from functional experiments, pharmacological manipulation and knock down of B2R suggest that B2R is the principle BK receptor in gliomas. This agrees with Lu et al. (Lu et al., 2010) suggesting BK as a ligand stimulating glioma migration.

Downstream of B2R activation is intracellular Ca²⁺-mobilization. Rat glioma C6 cells are sensitive to low dose of BK that selectively increases intracellular Ca²⁺-concentration (Wang and Liu, 2009). Similarly, we showed, based on the intracellular calcium change after BK treatment that human glioma cells also rapidly respond to a low dose of BK, and longer exposure to BK resulted in characteristic Ca²⁺-oscillations shown to be an underlying pattern for migration of cerebellar granule cells. As previously mentioned, the significance of calcium-dependent migration of astrocytoma (Ronde et al., 2000; Giannone et al., 2002) and neurons (Komuro and Rakic, 1996) has been reported by several groups. Interestingly, our data suggest a surprising, privileged role for BK-mediated Ca²⁺-signals in cell migration since other effectors, namely ACh and ATP, which both enhanced intracellular Ca²⁺ failed to influence cell migration. Most likely, signals downstream from Ca²⁺ are differentially activated and warrant further study. However, this data does not exclude the possibility that glioma cells could be sensitive to all of these ligands in situ.

Since BK is present in physiological conditions in the normal brain, its effects on normal glia have previously been studied and multiple effects of BK on glial cells have been described. For example, BK induces glutamate release (Parpura et al., 1994), MMP-9 expression and cell migration in normal astrocytes (Hsieh et al., 2008). Similar effects of BK were demonstrated on microglial migration (Ifuku et al., 2007). These studies together with the present findings suggest that BK acts broadly as a stimulator of migration in normal and malignant glia. The here described effects of BK on glioma invasion acting as chemo-tactic ligand attracting glioma cells to blood vessels had been previously unknown. Importantly, even in this system, BK appears to have multiple effects since our data also suggests an activation of MMPs in the presence of BK. Proteolysis is a crucial part of the invasion process, since tissue remodeling is necessary as cells invade through brain tissue. Both effects may work synergistically to enhance a more effective invasion. In addition, as BK enhances glioma cells motility, cells can become more prone and exposed to other chemo-tactic clues in their environment.

Invasion is a complex process that involves several coordinated phases: detachment from the primary tumor tissue, establishment new contacts with the environment, degradation and remodeling the extracellular matrix (ECM) and migration into healthy tissue (Rao, 2003). Once detached, glioma cells are exposed to the same signaling molecules as any other normal cell in the brain. They disperse along myelinated nerve fibers, subependymal layers and vasculature (Zagzag et al., 2008). However, the signals that attract them to and keep them on along these “highways” are not well understood. Therefore, the most interesting finding of these studies came from our slice invasion experiments. Our data suggest an important role for BK signaling in assuring that glioma cells find blood vessels to associate with. The number of cells attached to the blood vessels in the presence of BK dramatically increased when compared to control. Moreover, glioma cells invade deeper into the brain tissue when exposed to BK. This data implies that endothelial cells, by initiating BK production may provide an important cue for glioma cells to find blood vessels which is crucial for the cells to obtain sufficient nutrients to develop satellite tumors. This is clearly not the only signaling mechanism as even in the presence of a B2R antagonist some glioma cells still associate with blood vessels. Interestingly, the reduction in the number of glioma cells attached to blood vessels below was control level when slices were bathed either in the presence of BK with the antagonist, or the antagonist alone suggesting a disruption of endogenous BK signaling. This may involve interaction with normal brain cells as they also express B2R. For example, BK induces release of a number of signaling molecules from astrocytes such are glutamate, D-serine, ATP to name a few (Parpura et al., 1994; Montana et al., 2004; Martineau et al., 2008; Verderio and Matteoli, 2001). Any and/or all of these compounds can stimulate glioma migration (Lyons et al., 2007). If HOE-140 blocks B2R on astrocytes, the release would be blocked as well and the effect would be significantly reduced. While in vitro data suggest a direct effect of BK on glioma, the slice experiments imply that more complex signaling and cross talk between normal brain cells and glioma may be involved. One cannot distinguish between direct and indirect effects involving other brain cells in this study, but this question may warrant future studies.

Despite improvements in the diagnosis and treatment of patients with glial tumors, they remain the most common and least curable brain cancer in adults. The ability of glioma cells to infiltrate surrounding brain tissue, and ultimately escape current therapeutic interventions, could potentially be minimized using anti-invasive therapies. This study discloses a hitherto unknown effect of BK on glioma cells. Considering the strong effect of BK on glioma invasion and the prominent expression B2R that correlates with the grade of glioma, B2R emerges as an attractive therapeutic target. Developments of pharmaceutical approaches that target B2R, though for different condition, are well under way. In July 2008, Icatibant (Firazyr; Jerini, Berlin, Germany), a B2R antagonist, was granted market authorization by the European Commission for the symptomatic treatment of acute attacks of hereditary angioedema. (Bork et al., 2010). As a single therapeutic approach is proven to be ineffective in curing neoplasms, it is necessary to develop combined strategies to fight uncontrollable growth and dissemination. It is conceivable that B2R arises as one of the targets.

### REFERENCES

Abe, Y., Kayakiri, H., Satoh, S., Inoue, T., Sawada, Y., Inamura, N., Asano, M., Aramori, I., Hatori, C., Sawai, H., Oku, T. & Tanaka. H. (1998). A novel class of orally active non-peptide bradykinin B2 receptor antagonists. 4. Discovery of novel frameworks mimicking the active conformation. J. Med. Chem., 41, 4587-4598.
Asano, M., Inamura, N., Hatori, C., Sawai, H., Fujiwara, T., Katayama, A., Kayakiri, H., Satoh, S., Abe, Y., Inoue, T., Sawada, Y., Nakahara, H., Oki, T. & Okuhara, M. (1997). The identification of an orally active, nonpeptide bradykinin B2 receptor antagonist, FR173657. Br. J. Pharmacol., 120, 617-624.
Bordey A, Sontheimer H, Trouslard J (2000) Muscarinic activation of BK channels induces membrane oscillations in glioma cells and leads to inhibition of cell migration. J Mem Biol 176: 31-40.
Bork K, Yaosothan U, Kirckpatrick, P (2010) Icatibant. Nature Rewievs Drug Discovery, 801-802. Ref Type: Report
Coller K E, Berger K L, Heaton N S, Cooper J D, Yoon R, Randall G. (2009) RNA interference and single particle tracking analysis of hepatitis C virus endocytosis. PLoS Pathog 5(12):e1000702.
Cote J, Savard M, Bovenzi V, Dubuc C, Tremblay L, Tsanaclis A M, Fortin D, Lepage M, Gobeil F, Jr. (2010) Selective tumor blood-brain barrier opening with the kinin B2 receptor agonist [Phe(8)psi(CH(2)NH)Arg(9)]-BK in a F98 glioma rat model: an MRI study. Neuropeptides 44: 177-185.
Couture R, Harrisson M, Vianna R M, and Cloutier F. (2001) Kinin receptors in pain and inflammation. Eur J. Pharmacol. 429(1-3):161-76.
Ewert S, Johansson B, Holm M, Helander H F, Fandriks L (2003) The bradykinin BK2 receptor mediates angiotensin II receptor type 2 stimulated rat duodenal mucosal alkaline secretion. BMC Physiol 3: 1.
Fabian A, Fortmann T, Bulk E, Bomben V C, Sontheimer H, Schwab A. (2010) Chemotaxis of MDCK-F cells toward fibroblast growth factor-2 depends on transient receptor potential canonical channel. Pflugers Arch. [Epub ahead of print]
Friedberg M H, Glantz M J, Klempner M S, Cole B F, Perides G (1998) Specific matrix metalloproteinase profiles in the cerebrospinal fluid correlated with the presence of malignant astrocytomas, brain metastases, and carcinomatous meningitis. Cancer 82: 923-930.
Giannone G, Ronde P, Gaire M, Haiech J, Takeda K (2002) Calcium oscillations trigger focal adhesion disassembly in human U87 astrocytoma cells. J Biol Chem 19; 277: 26364-26371.
Habela C W, Ernest N J, Swindall A F, Sontheimer H (2009) Chloride accumulation drives volume dynamics underlying cell proliferation and migration. J Neurophysiol. 101: 750-757.
Hall J, M. (1992) Bradykinin receptors: pharmacological properties and biological roles. Pharmac. Ther. 56, 131-190.
Hess J. F., Borkowski J. A., Young G. S., Strader C. D. and Ransom R. W. (1992) Cloning and pharmacological characterization of a human bradykinin (BK-2) receptor. Biochem. biophys. Res. Commun. 184, 260-268.
Higashida H, Yokoyama S, Hoshi N, Hashii M, Egorova A, Zhong Z G, Noda M, Shahidullah M, Taketo M, Knijnik R, Kimura Y, Takahashi H, Chen X L, Shin Y, Zhang J S (2001) Signal transduction from bradykinin, angiotensin, adrenergic and muscarinic receptors to effector enzymes, including ADP-ribosyl cyclase. Biol Chem 382: 23-30.
Hsieh H L, Wu C Y, Yang C M (2008) Bradykinin induces matrix metalloproteinase-9 expression and cell migration through a PKC-delta-dependent ERK/Elk-1 pathway in astrocytes. Glia 56: 619-632.
Ifuku M, Farber K, Okuno Y, Yamakawa Y, Miyamoto T, Nolte C, Merrino V F, Kita S, Iwamoto T, Komuro I, Wang B, Cheung G, Ishikawa E, Ooboshi H, Bader M, Wada K, Kettenmann H, Noda M (2007) Bradykinin-induced microglial migration mediated by B1-bradykinin receptors depends on Ca²⁺ influx via reverse-mode activity of the Na⁺/Ca²⁺ exchanger. J Neurosci 27: 13065-13073.
Ishiuchi S, Tsuzuki K, Yoshida Y, Yamada N, Hagimura N, Okado H, Miwa A, Kurihara H, Nakazato Y, Tamura M, Sasaki T, Ozawa S (2002) Blockage of Ca(²⁺)-permeable AMPA receptors suppresses migration and induces apoptosis in human glioblastoma cells. Nat Med 8: 971-978.
Jacobelli J, Bennett F C, Pandurangi P, Tooley A J, Krummel (2009) MF Myosin-IIA and ICAM-1 regulate the interchange between two distinct modes of TI cell migration. J Immunol 182: 2041-2050.
Joseph K, Kaplan A P (2005) Formation of bradykinin: a major contributor to the innate inflammatory response. Adv Immunol 86: 159-208.
Kaba S E, Kyritsis A P (1997) Recognition and management of gliomas. Drugs 53: 235-244.
Komuro H, Rakic P (1996) Intracellular Ca²⁺ fluctuations modulate the rate of neuronal migration. Neuron 17: 275-285.
Kronlage M, Song J, Sorokin L, Isfort K, Schwerdtle T, Leipziger J, Robaye B, Conley P B, Kim H C, Sargin S, Schdn P, Schwab A, Hanley P J. (2010) Autocrine purinergic receptor signaling is essential for macrophage chemotaxis. Sci Signal. 3(132):ra55.
Laerum O D, Bjerkvig R, Steinsvag S K, de Ridder L (1984) Invasiveness of primary brain tumors. Cancer Metastasis Reviews 3: 223-236.
Lu D Y, Leung Y M, Huang S M, Wong K L (2010) Bradykinin-induced cell migration and COX-2 production mediated by the bradykinin B1 receptor in glioma cells. J Cell Biochem 110: 141-150.
Lyons S A, Chung W J, Weaver A K, Ogunrinu T, Sontheimer H (2007) Autocrine glutamate signaling promotes glioma cell invasion. Cancer Res 67: 9463-9471.
Martineau M, Galli T, Baux G, Mothet J P (2008) Confocal imaging and tracking of the exocytotic routes for D-serine-mediated gliotransmission. Glia 56: 1271-1284.
Martins A H, Alves J M, Trujillo C A, Schwindt T T, Barnabe G F, Motta F L, Guimaraes A O, Casarini D E, Mello L E, Pesquero J B, Ulrich H (2008) Kinin-B2 receptor expression and activity during differentiation of embryonic rat neurospheres. Cytometry A 73: 361-368.
McEachern A. E., Shelton, E. R., Bhakta, S., Obernolte. R., Bach. C., Zuppan. P., Fujisaki, J., Aldrich, R. W. and Jarnagin, K. (1991) Expression cloning of a rat B2 bradykinin receptor. Proc. Natn. Acad. Sci. U.S.A. 88:7724
Montana V, Ni Y, Sunjara V, Hua X, Parpura V (2004) Vesicular glutamate transporter-dependent glutamate release from astrocytes. J Neurosci 24: 2633-2642.
Moreau M E, Garbacki N, Molinaro G, Brown N J, Marceau F, Adam A (2005) The kallikrein-kinin system: current and future pharmacological targets. J Pharmacol Sci 99: 6-38.
Parpura V, Basarsky T A, Liu F, Jeftinija K, Jeftinija S, Haydon P G (1994) Glutamate-mediated astrocyte-neuron signalling. Nature 369: 744-747.
Raidoo D M, Sawant S, Mahabeer R, Bhoola K D (1999) Kinin receptors are expressed in human astrocytic tumour cells. Immunopharmacology 43: 255-263.
Rao J S (2003) Molecular mechanisms of glioma invasiveness: the role of proteases. Nat Rev Cancer 3: 489-501.
Ratajczak M Z, Zuba-Surma E, Kucia M, Reca R, Wojakowski W, Ratajczak J (2006) The pleiotropic effects of the SDF-1-CXCR4 axis in organogenesis, regeneration and tumorigenesis. Leukemia 20: 1915-1924.
Reetz G, Reiser G (1996) [Ca²⁺] oscillations induced by bradykinin in rat glioma cells associated with Ca²⁺ store-dependent Ca²⁺ influx are controlled by cell volume and by membrane potential. Cell Calc 19: 143-156.
Ronde P, Giannone G, Gerasymova I, Stoeckel H, Takeda K, Haiech J (2000) Mechanism of calcium oscillations in migrating human astrocytoma cells. Biochim Biophys Acta 20; 1498: 273-280.
Salvino, J. M., Seoane, P. R., Douty, B. D., Awad, M. M. A., Dolle, R. E., Houck, W. T., Faunce, D. M., Sawutz, D. G. (1993). Design of potent non-peptide competitive antagonists of the human bradykinin B2 receptor. J. Med. Chem., 36, 2583-2584.
Sarin H, Kanevsky A S, Fung S H, Butman J A, Cox R W, Glen D, Reynolds R, Auh S (2009) Metabolically stable bradykinin B2 receptor agonists enhance transvascular drug delivery into malignant brain tumors by increasing drug half-life. J Transl Med 7: 33.
Uchida M, Chen Z, Liu Y, Black K L (2002) Overexpression of bradykinin type 2 receptors on glioma cells enhances bradykinin-mediated blood-brain tumor barrier permeability increase. Neurol Res 24: 739-746.
Verderio C, Matteoli M (2001) ATP mediates calcium signaling between astrocytes and microglial cells: modulation by IFN-gamma. J Immunol 166: 6383-6391.
Wang Y B, Liu Y H (2009) Bradykinin selectively modulates the blood-tumor barrier via calcium-induced calcium release. J Neurosci Res 87: 660-667.
Wang Y B, Peng C, Liu Y H (2007) Low dose of bradykinin selectively increases intracellular calcium in glioma cells. J Neurol Sci 258: 44-51.
Weaver A K, Bomben V C, Sontheimer H (2006) Expression and function of calcium-activated potassium channels in human glioma cells. Glia 54: 223-233.
Xia C Y, Zhang Z, Xue Y X, Wang P, Liu Y H (2009) Mechanisms of the increase in the permeability of the blood-tumor barrier obtained by combining low-frequency ultrasound irradiation with small-dose bradykinin. J Neurooncol 94: 41-50.
Yang W H, Chang J T, Hsu S F, Li T M, Cho D Y, Huang C Y, Fong Y C, Tang C H (2010) Bradykinin enhances cell migration in human chondrosarcoma cells through BK receptor signaling pathways. J Cell Biochem 109: 82-92.
Zagzag D, Esencay M, Mendez O, Yee H, Smirnova I, Huang Y, Chiriboga L, Lukyanov E, Liu M, Newcomb E W (2008) Hypoxia- and vascular endothelial growth factor-induced stromal cell-derived factor-1alpha/CXCR4 expression in glioblastomas: one plausible explanation of Scherer's structures. Am J Pathol 173: 545-560.
Zhang Z, Xia C, Xue Y, Liu Y (2009) Synergistic effect of low-frequency ultrasound and low-dose bradykinin on increasing permeability of the blood-tumor barrier by opening tight junction. J Neurosci Res 87: 2282-2289.
Zhao Y, Xue Y, Liu Y, Fu W, Jiang N, An P, Wang P, Yang Z, Wang Y (2005) Study of correlation between expression of bradykinin B2 receptor and pathological grade in human gliomas. Br J Neurosurg 19: 322-326.

### SEQUENCES

The following are provided in the attached sequence listing.

SEQ ID NO: 1—canonical human bradykinin.
SEQ ID NO: 2—canonical human bradykinin-2-receptor.
SEQ ID NO: 3—canonical chimpanzee bradykinin-2-receptor.
SEQ ID NO: 4—canonical wolf bradykinin-2-receptor.
SEQ ID NO: 5—canonical cattle bradykinin-2-receptor.
SEQ ID NO: 6—canonical mouse bradykinin-2-receptor.
SEQ ID NO: 7—canonical rat bradykinin-2-receptor.
SEQ ID NO: 8—canonical chicken bradykinin-2-receptor.
SEQ ID NO: 9—canonical zebrafish bradykinin-2-receptor.
SEQ ID NO: 10—canonical human GFAP.
SEQ ID NO: 11—canonical chimpanzee GFAP.
SEQ ID NO: 12—canonical wolf GFAP.
SEQ ID NO: 13—canonical cattle GFAP.
SEQ ID NO: 14—canonical mouse GFAP.
SEQ ID NO: 15—canonical rat GFAP.
SEQ ID NO: 16—canonical chicken GFAP.
SEQ ID NO: 17—canonical zebrafish GFAP.

### CONCLUSIONS

The present disclosure shows that glioma cells isolated from patient biopsies express B2R whose activation causes intracellular Ca²⁺-oscillations. Through time-lapse video-microscopy experiments, the present disclosure shows that BK significantly enhances glioma cell migration/invasion and that BK acts as a chemo-attractant guiding glioma cells toward blood vessels in acute rat brain slices. The number of cells associated with blood vessels is decreased when B2R is either pharmacologically inhibited or B2R eliminated through shRNA knockdown. The present disclosure shows that bradykinin, acting via B2R, acts as an important signal directing the invasion of glioma cells toward blood vessels. B2R antagonists, including currently available clinically approved B2R antagonists, can be used therapeutics for the treatment of cancer, including, but not limited to, glioma.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein.

We claim:

1. A composition comprising a therapeutically effective amount of an inhibitor selected from the group consisting of inhibitor of bradykinin (BK) and an inhibitor of the bradykinin-2-receptor (B2R).

2. (canceled)

3. A method of modulating migration of a glial cell, said method comprising contacting the cell with a modulator selected from the group consisting of: a modulator of BK and a modulator of B2R.

4. The method of claim 3, wherein the modulator is an activator.

5. The method of claim 3, wherein the modulator is an inhibitor.

6. The method of claim 5, wherein the inhibitor is an inhibitor of BK.

7. The method of claim 5, wherein the inhibitor is an inhibitor of B2R.

8. The method of claim 5, wherein the inhibitor is a nucleic acid inhibitor, an antibody, or an antibody fragment.

9. The method of claim 7 wherein the inhibitor is not an inhibitor of the bradykinin-1-receptor.

10. The method of claim 7 wherein the inhibitor is selected from the group consisting of: bradyzide, HOE-140, icatibant, FR173657, FR193517, a tautomer of any of the foregoing, and a pharmaceutically acceptable salt of any of the foregoing.

11. The method of claim 6 wherein the inhibitor is an antibody or antibody fragment that recognizes BK.

12. The method of claim 7 wherein the inhibitor is an antibody or antibody fragment that recognizes B2R.

13. The method of claim 5, wherein the inhibitor is an siRNA.

14. A method of determining the invasive potential of a cancer cell, said method comprising:
(a) measuring a property of the cell, said property selected from the group consisting of B2R activity and B2R expression; and
(b) comparing the property to a baseline value;
wherein the cell is determined to have increased invasive potential if the property is higher than the baseline value.

15. The method of claim 14, comprising measuring a second property of the cell selected from the group consisting of the activity of glial fibrillary acidic protein (GFAP) or the expression of GFAP, wherein the cell is determined to have increased invasive potential if the second property is decreased compared to a second baseline.

16. The method of claim 14, wherein one or both of the first property and the second property is measured by immunostaining.

17. The method of claim 14, wherein the property of the cell is measured in vitro.

18. The method of claim 14, comprising obtaining a sample from a subject, the sample comprising the cell.

19. The method of claim 14, wherein the baseline value is a value corresponding to at least one of a non-cancerous cell and a non-invasive cancer cell.

20. A method of treating or preventing the migration of a cancer in a subject in need thereof, said method comprising administering the composition of claim 1 to said subject.

21. A kit for determining the invasive potential of a cancer cell in vitro, said kit comprising a means to measure a property of the cell, said property selected from the group consisting of B2R activity and B2R expression.

22. The kit of claim 21, wherein the means to measure the property of the cell is an antibody preparation.

23. The kit of claim 22, wherein the antibody preparation comprises an antibody or an antibody fragment that recognizes B2R.

24. The kit of claim 21, wherein the antibody preparation comprises a reporter conjugated to an antibody or an antibody fragment.

25. The composition of claim 1, wherein the inhibitor is an inhibitor of BK.

26. The composition of claim 1, wherein the inhibitor is an inhibitor of B2R.

27. The composition of claim 1, wherein the inhibitor is a nucleic acid inhibitor, an antibody, or an antibody fragment.

28. The composition of claim 26, wherein the inhibitor is not an inhibitor of the bradykinin-1-receptor.

29. The composition of claim 26, wherein the inhibitor is selected from the group consisting of: bradyzide, HOE-140, icatibant, FR173657, FR193517, a tautomer of any of the foregoing, and a pharmaceutically acceptable salt of any of the foregoing.

30. The composition of claim 25, wherein the inhibitor is an antibody or antibody fragment that recognizes BK.

31. The composition of claim 26, wherein the inhibitor is an antibody or antibody fragment that recognizes B2R.

32. The composition of claim 1, wherein the inhibitor is an siRNA.


SEQUENCE LISTING (17 sequences)

SEQ ID NO: 1 - protein, 9 aa, Homo sapiens
RPPGFSPFR

SEQ ID NO: 2 - protein, 391 aa, Homo sapiens
MFSPWKISMFLSVREDSVPTTASFSADMLNVTLQGPTLNGTFAQSKCPQVEWLGWLNTIQPPFLWVLFVLATLENIFVLSVFCLHKSSCTVAEIYLGNLAAADLILACGLPFWAITISNNFDWLFGETLCRVVNAIISMNLYSSICFLMLVSIDRYLALVKTMSMGRMRGVRWAKLYSLVIWGCTLLLSSPMLVFRTMKEYSDEGHNVTACVISYPSLIWEVFTNMLLNVVGFLLPLSVITFCTMQIMQVLRNNEMQKFKEIQTERRATVLVLVVLLLFIICWLPFQISTFLDTLHRLGILSSCQDERIIDVITQIASFMAYSNSCLNPLVYVIVGKRFRKKSWEVYQGVCQKGGCRSEPIQMENSMGTLRTSISVERQIHKLQDWAGSRQ

SEQ ID NO: 3 - protein, 391 aa, Pan troglodytes
MFSPWKIPMFLSVREDSVPTTASFSTDMLNVTLQGPTLNGTFAQSKCPQVEWLGWLNTIQPPFLWVLFVLATLENIFVLSVFCLHKSSCTVAEIYLGNLAAADLILACGLPFWAITISNNFDWLFGETLCRVVNAIISMNLYSSICFLMLVSIDRYLALVKTMSMGRMRGVRWAKLYSLVIWGCTLLLSSPMLVFRTMKEYSDEGHNVTACVISYPSLIWEVFTNMLLNVVGFLLPLSVITFCTMQIMQVLRNNEMQKFKEIQTERRATVLVLVVLLLFIICWLPFQISTFLDTLHRLGILSSCQDERIIDVITQIASFMAYSNSCLNPLVYVIVGKRFRKKSWEVYQGVCQKGGCRSEPIQMENSMGTLRTSISVERQIHKLQDWAGSRQ

SEQ ID NO: 4 - protein, 392 aa, Canis lupus
MFSAWKRPMFLSFHEDPVPTTASLSTEMFNSTSQDLMPTLNGTLPSPCVYPEWWNWLNTIQAPFLWVLFILAALENLFVLSIFCLHKSSCTVAEIYLGNLALADLILASGLPFWAITIANNFDWLFGEVLCRVVNTMLYMNLYSSICFLMLVSIDRYLALVKTMSMGRMRGVRWAKLYSLVIWGCTLLLSSPMLAFRTMKEYRDEGYNVTACVIIYPSRTWEVFTNVLLNFVGFLLPLTVITFCTVQIMQVLRNNEMQKFKEIQTERKATVLVLAVLLLFVICWLPFQISTFLDTLLRLDILSGCRHEHLVDVFTQIASYVAYSNSCLNPLVYVIVGKRFRKKSREVFRGLCQKGGCVLESNKMDNSMGTLRTSVSVERQIHKLPEWAENSQ

SEQ ID NO: 5 - protein, 388 aa, Bos taurus
MFSAWRRPMFLSIHEDTVPTTASFGAEMFNLTSQVLEPALNGTLPEGSSCFQSDLWNWLNTIQPPFLWILFLLAALENTFVLSVFCLHKSSCTVAEIYLGNLAVADLILACGLPFWAITIANNFDWLFGEALCRVVNTILYMNLYSSIYFLMLVSIDRYLALVKTMSMGRMRGVRWAKLYSLVIWGCALLLSSPMLAFRTMQEYNAEGHNVTACVINYPSHSWEVFTNILLNSVGFLLPLSVITFCTVQIMQVLRNNEMQKFKEIQTERKATLLVLAVLLLFVVCWLPFQISTFLDTLLRLHVLSGCWDEYVIDIFTQIASFVAYSNSCLNPLVYVIVGKRFRKKSQEVYARLCRPGGCGSAEPSQTENSMGTLRTSISVERNIHKLQ

SEQ ID NO: 6 - protein, 366 aa, Mus musculus
MFNVTTQVLGSALNGTLSKDNCPDTEWWSWLNAIQAPFLWVLFLLAALENLFVLSVFFLHKNSCTVAEIYLGNLAAADLILACGLPFWAITIANNFDWVFGEVLCRVVNTMIYMNLYSSICFLMLVSIDRYLALVKTMSMGRMRGVRWAKLYSLVIWGCTLLLSSPMLVFRTMREYSEEGHNVTACVIVYPSRSWEVFTNVLLNLVGFLLPLSVITFCTVRILQVLRNNEMKKFKEVQTERKATVLVLAVLGLFVLCWVPFQISTFLDTLLRLGVLSGCWDEHAVDVITQISSYVAYSNSGLNPLVYVIVGKRFRKKSREVYRVLCQKGGCMGEPVQMENSMGTLRTSISVERQIHKLQDWAGKKQ

SEQ ID NO: 7 - protein, 366 aa, Rattus norvegicus
MFNITTQALGSAHNGTFSEVNCPDTEWWSWLNAIQAPFLWVLFLLAALENIFVLSVFCLHKTNCTVAEIYLGNLAAADLILACGLPFWAITIANNFDWLFGEVLCRVVNTMIYMNLYSSICFLMLVSIDRYLALVKTMSMGRMRGVRWAKLYSLVIWSCTLLLSSPMLVFRTMKDYREEGHNVTACVIVYPSRSWEVFTNMLLNLVGFLLPLSIITFCTVRIMQVLRNNEMKKFKEVQTEKKATVLVLAVLGLFVLCWFPFQISTFLDTLLRLGVLSGCWNERAVDIVTQISSYVAYSNSCLNPLVYVIVGKRFRKKSREVYQAICRKGGCMGESVQMENSMGTLRTSISVDRQIHKLQDWAGNKQ

SEQ ID NO: 8 - protein, 381 aa, Gallus gallus
MITITTENVTQLYNVTATQEFTISPAEFHNNSSVHQMDEYKCINPDTWKWLQTFQPGFLWIIFILGTIENAFVLIVLYFHKSRCSVAEIYLANMAFADLMLVCSLPFWAINISNGFQWPFGLFLCKAVNTINYMNSYSSIYFLTLVSIDRYLALVKTMSLGRMRRTACAKWNSLIIWMCALLICSPTMVFRNLQYFKEYNITACSLDYPTPYWHPANNCLLNAVGFMIPLCIITYCTTQIIKALQSNKSQKLKLIQTERKATLLVLAVLLLFVICWLPFQISTLIDTVCYLAGNLKCLEDINDIVTQMAIYCAFSNSCLNPILYVIVGKHFQKKAVELFKDLIPQRCRKSESVKIENSQDTLRTSISSEHLRKKSVLPLSQ

SEQ ID NO: 9 - protein, 356 aa, Danio rerio
MDPEIQLTTSSSILSTIPSTNNLTNATQCPHWEVWDWLYVMQPAYMFIICVLGIIGNIFVLLVFSLHKKACTVAEIYLGNLAAADLLLVSCLPFWAINIANEFNWEFGSAMCRLVNTGIKMNMLCSIYFLVLVSTDRYVALVHALSRGRMRRPRYAKLNCIAVWCFGLILNIPTLHFRDIKFIPELNITACILDYPNPNIGLICDILLMIIGFIIPILVISYCTLKIIRALHEQVVDRFNAENTERKATILVLVVLMVFLLCWVPFHLVTLMDVLMRFGVFSGCTFEAGLDISNQIFTYLALSNSVLNPILYVIVGKNFRKKVKELMKQLNEKKADSTSGSTRSQLSSTLKTFTTY

SEQ ID NO: 10 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 11 - protein, 502 aa, Pan troglodytes
MQELACLGSSASGHSDLSGVRGALPIAGLRPNPTPPGYARGFCQGHPGIASPAHSFIKPSHPRSEQSQSRMERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 12 - protein, 433 aa, Canis lupus
MERRRVASAARRSYVYVSSWDMAGGGPGSGRRLGPGPRPSVARMPLPPTRVDFSLAAALNAGFKETRASERAEMMELNDRFASYIEKVRFLEQQNKALAAELNQLRAKEPTKLADVYQAELRELRLRLDQLTANSARLEVERDNLAQDLGTLRQKFQDETNLRLEAENNLASYRQEADEATLARLDLERKIESLEEEIRFLRKIHDEEVQELQEQLARQQVHVELDVAKPDLTAALREIRTQYEAMASSNMHEAEEWYRSKFADLTDAAARNAELLRQAKHEANDYRRQLQTLTCDLESLRGTNESLERQMREQEERHAREAASYQEALARLEEEGQNLKDEMARHLQEYQDLLNVKLALDIEIATYRKLLEGEENRITIPVQTFSNLQIRETSLDTKSVSEGHLKRNIVVKTVEMRDGEVIKESKQEHKEVM

SEQ ID NO: 13 - protein, 428 aa, Bos taurus
MERRRVTSATRRSYVSSSEMVVGGRRLGPGTRLSLARMPPPLPARVDFSLAGALNSGFKETRASERAEMMELNDRFASYIEKVRFLEQQNKALAAELNQLRAKEPTKLADVYQAELRELRLRLDQLTANSARLEVERDNLAQDLGTLRQKLQDETNQRLEAENNLAAYRQEADEATLARLDLERKIESLEEEIRFLRKIHEEEVRELQEQLAQQQVHVEMDVAKPDLTAALREIRTQYEAVASSNMHEAEEWYRSKFADLNDAAARNAELLRQAKHEANDYRRQLQALTCDLESLRGTNESLERQMREQEERHAREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 14 - protein, 430 aa, Mus musculus
MERRRITSARRSYASETVVRGLGPSRQLGTMPRFSLSRMTPPLPARVDFSLAGALNAGFKETRASERAEMMELNDRFASYIEKVRFLEQQNKALAAELNQLRAKEPTKLADVYQAELRELRLRLDQLTANSARLEVERDNFAQDLGTLRQKLQDETNLRLEAENNLAAYRQEADEATLARVDLERKVESLEEEIQFLRKIYEEEVRELREQLAQQQVHVEMDVAKPDLTAALREIRTQYEAVATSNMQETEEWYRSKFADLTDAASRNAELLRQAKHEANDYRRQLQALTCDLESLRGTNESLERQMREQEERHARESASYQEALARLEEEGQSLKEEMARHLQEYQDLLNVKLALDIEIATYRKLLEGEENRITIPVQTFSNLQIRETSLDTKSVSEGHLKRNIVVKTVEMRDGEVIKDSKQEHKDVVM

SEQ ID NO: 15 - protein, 430 aa, Rattus norvegicus
MERRRITSARRSYASSETMVRGHGPTRHLGTIPRLSLSRMTPPLPARVDFSLAGALNAGFKETRASERAEMMELNDRFASYIEKVRFLEQQNKALAAELNQLRAKEPTKLADVYQAELRELRLRLDQLTTNSARLEVERDNLTQDLGTLRQKLQDETNLRLEAENNLAVYRQEADEATLARVDLERKVESLEEEIQFLRKIHEEEVRELQEQLAQQQVHVEMDVAKPDLTAALREIRTQYEAVATSNMQETEEWYRSKFADLTDVASRNAELLRQAKHEANDYRRQLQALTCDLESLRGTNESLERQMREQEERHARESASYQEALARLEEEGQSLKEEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 16 - protein, 422 aa, Gallus gallus
MESRRLSYGRRFGPTGSAWSPTERPRPRGAHPAPRTSGRMDFSLANVLNSEFRETRTNEKVEMMELNDRFASYIEKVRLLEQRNKVLVLELNRAREQQPSRLGDVYQEELRELRRRVELLGTAKARAEVQRDGLAEELGSLREKLQQEVNLRLEAESTLAAYRQDVDAAALARLDLERRVGSLQDEVAFLQKVHEEELRELQEQLAQHQVHVEVDASKPDLTAALRDIRTQYEAMAASNMQETEEWYKSKFADLTDAAARHAEALRAAKHEANEYRRQLQALTCDLEALRGSNESLERQLRELEERYALETSAYQDTVGRLEEDIHSLKEEMGRHLQEYQDLLNVKLALDIEIATYRKLLEGEESRITVPVQSFSNLQIRETSLDTKSVSEAHVKRSIVVKTVETRDGEVIKESKQEHKEVV

SEQ ID NO: 17 - protein, 444 aa, Danio rerio
MESQRSFSSYRKRFGTPGGSPSVGVTSRHSTGRLSLHSSPRHLTSSPLTLSTSRLSLGGERLDFSADSLLKAQYRETRTNEKVEMMGLNDRFASYIEKVRFLEQQNKMLVAELNQLRGKEPSRLGDIYQEELRELRRQVDGLNAGKARLEIERDNLASDLGTLKQRLQDETALRQEAENNLNTFRQDVDEAALNRVQLERKIEALQDEINFLKKVHEEEMRELHEQLMAQQVHVDLDVSKPDLTAALKEIRAQFEAMANSNMQETEEWYRSKFADLTDAANRNGEALRQAKQEANDYRRQIQGLTCDLESLRGSNESLERQLKEMEERFAIETAGYQDTVARLEDEIQMLKEEMARHLQEYQDLLNVKLALDIEIATYRKLLEGEESRITVPVQNFTNLQFRDTSMDTKLTPEAHVKRSIVVRTVETRDGEIIKESTTERKDLP